(19) United States
(12) Patent Application Publication
(10) Pub. No.: US 2024/0123121 A1
(43) Pub. Date: Apr. 18, 2024

(54) BIOPRINTED SOFT TISSUE REINFORCEMENT SCAFFOLD

(71) Applicants: ETHICON, INC., Raritan, NJ (US); T&R BIOFAB CO. LTD., Siheung-si (KR)

(72) Inventors: Orquidea GARCIA, St. Charles, IL (US); Donal CONROY, Dublin (IE); Songwan JIN, Seongnam-si (KR); Inho KIM, Seongnam-si (KR); Songho KIM, Seongnam-si (KR); Chaehyeon RYU, Seongnam-si (KR); Jinhyung SHIM, Seongnam-si (KR); Hyunjung KIM, Seongnam-si (KR); Changhwan KIM, Seongnam-si (KR)

(21) Appl. No.: 18/470,567

(22) Filed: Sep. 20, 2023

Related U.S. Application Data
(60) Provisional application No. 63/376,658, filed on Sep. 22, 2022.

(51) Int. Cl. A61L 27/36; A61L 27/28; A61L 27/54; A61L 27/56; B33Y 80/00

(52) U.S. Cl. CPC A61L 27/3633; A61L 27/28; A61L 27/54; A61L 27/56; B33Y 80/00; A61L 2420/02; A61L 2430/34

(57) ABSTRACT

The disclosure encompasses systems, compositions, and methods for use in vivo, including for reinforcement of soft tissue in an individual. The systems, compositions, and methods may utilize three-dimensionally printed scaffolds comprising at least a polymer scaffold and an extracellular matrix component(s), including comprised on the scaffold. The polymer scaffold may comprise particular unit cell structures of a specific design and patterns of alternating configurations of the unit cell structures.

This application claims priority to U.S. Provisional Application Ser. No. 63/376,658, filed Sep. 22, 2022, which is incorporated by reference herein in its entirety.

### BACKGROUND

This disclosure relates at least to the fields of chemistry, biology, biologically compatible structures, wound care, and medicine, including compositions and materials used to form bioscaffold structures that can be utilized in in an individual, such as for soft tissue reinforcement and/or regeneration. There are a variety of materials available to provide tissue reinforcement and/or fill-in resected tissue to reconstruct the native shape of soft tissue.

### SUMMARY OF THE DISCLOSURE

Embodiments of the disclosure include systems, methods, and compositions related to soft tissue regeneration, reinforcement, and/or repair. In various embodiments, the disclosure concerns particular structures for regeneration, reinforcement, and/or repair of soft tissue, wherein the structures provide structural and therapeutic elements to facilitate such activities. In particular embodiments, the disclosure relates to scaffolds that are configured to mechanically support soft tissue while also delivering biological molecules that enhance at least regeneration and repair of the soft tissue, and in specific cases the scaffold elicits minimal or no immunogenic response in the recipient individual. In specific embodiments, the disclosure provides scaffold compositions manufactured of materials that minimize immunogenic response in a host while providing a structural framework for tissue regrowth. In embodiments, the scaffold bestows two-fold benefits to the recipient individual by providing: (1) a physical structure comprised of defined unit cell structures to provide support, yet comprised of a suitable polymer to impart appropriate strength and flexibility for use in vivo; and (2) a therapeutic component as a coating (and/or within the physical structure) to enable enhanced cell and/or tissue proliferation and/or infiltration at a soft tissue site in need. In some embodiments, the scaffold acts as a delivery component to deliver the therapeutic component(s). In particular embodiments, the scaffold utilizes a particular pattern to recapitulate native mechanical tissue properties that other synthetic scaffolds cannot. In embodiments, three dimensional (3D) printing produces the scaffold.

Particular embodiments provide for a scaffold comprised of one or more biodegradable polymers and one or more extracellular matrix materials in which the scaffold is configured of rows of unit cell structures or unit patterns of a repeating, particular design. In embodiments, the design of the unit cell structure or unit pattern comprises a particular shape, including a shape that is generally the structure of the capital letter “I.” In specific embodiments, the scaffold comprises rows of unit cell structures or unit patterns of the capital letter “I” in perpendicularly alternating configurations.

Embodiments of the disclosure provide an artificial support structure comprising one or more biodegradable polymers and one or more extracellular matrix materials, the support structure further comprising a plurality of unit patterns, each unit pattern comprising a plurality of filaments, arranged continuously, symmetrically, and regularly thereon, each unit pattern being constituted of edges of a closed shape to thus form a pore at the inside thereof, wherein the plurality of unit patterns is connected to thus have intersection points with one another, and the number of intersection points is the same as the number of edges passing the intersection points. In specific embodiments, the one or more extracellular matrix materials comprises Collagen I. The at least one unit pattern in certain embodiments has a diameter of between about 200 microns to about 3.5 mm. The at least one unit pattern has a diameter of between about 1.5 mm to about 3 mm or about 1.782 mm to about 2.97 mm, in certain aspects. The biodegradable polymer material may further comprise one or more extracellular matrix materials, in specific embodiments, such as Collagen I.

In particular embodiments, the plurality of connected unit patterns forms a substantially planar sheet. The plurality of connected unit patterns may form a three-dimensional macrostructure, in specific aspects, and the artificial support structure has a thickness between about 0.5 mm to about 1.5 mm, about 0.7 mm to about 1.3 mm, or about 0.9 mm to about 1.1 mm, in various embodiments. In some cases, the artificial support structure comprises, consists or, or consists essentially of between 1 and 5 layers, and the layers may have thicknesses of between about 0.10 mm to about 0.3 mm, about 0.15 mm to about 0.25 mm, or about 0.18 mm to about 0.22 mm. In some embodiments, one or more filaments in the plurality of filaments have a diameter of less than about 550, 500, or 400 microns.

In some embodiments, the four-unit patterns are connected to have four intersection points with one another and have four edges passing or crossing at the four intersection points, and a space surrounded with the four-unit patterns has the same or similar shape as or to each unit pattern. Each unit pattern is an uppercase “I” of the English alphabet, in specific embodiments, and the short edges of the edges of the closed shape of each unit pattern have the same length as one another, and long edges thereof have the same length as one another, so that the space surrounded with the four-unit patterns has the same shape as each unit pattern, in some embodiments. In the closed shape, a length ratio of the short edge to the long edge is 1:3, in specific cases.

In various embodiments, the artificial support structure has an ultimate tensile strength of between about 4 MPa to about 5 MPa, about 4.05 MPa to about 4.7 MPa, or about 4.12 MPa to about 4.50 MPa. In some embodiments, the artificial support structure has a modulus of elasticity of about 2.8 MPa to about 4.2 MPa, about 3.00 MPa to about 4.10 MPa, or about 3.06 Mpa to about 4.00 Mpa. In certain embodiments, the artificial support structure has a suture retention strength of about 20 N to about 26 N, about 21 N to about 25 N, or about 22.03 N to about 24.27 N. In particular embodiments, the artificial support structure has a burst strength of about 140 N to about 170 N, about 145 N to about 163 N, or about 147.14 N to about 161.26 N. In various embodiments, the artificial support structure has a tear resistance of about 18 N to about 26 N, about 19 N to about 25 N, or about 19.87 N to about 24.92 N.

Embodiments of the disclosure encompass an artificial support structure comprising one or more biodegradable polymers and one or more extracellular matrix materials (such as Collagen I), the support structure further comprising a plurality of unit patterns, each unit pattern comprising a plurality of filaments, arranged repeatedly to constitute columns or rows symmetrical to one another, each unit pattern being constituted of edges of a closed shape to thus form a pore at the inside thereof, wherein the columns or rows along which the plurality of unit patterns are arranged repeatedly have an Eulerian trail. In particular embodiments, at least one unit pattern has a diameter of between about 200 microns to about 3.5 mm, about 1.5 mm to about 3 mm, or about 1.782 mm to about 2.97 mm. In certain embodiments, the biodegradable polymer material further comprises one or more extracellular matrix materials, such as Collagen I.

In certain embodiments, the plurality of connected unit patterns form a substantially planar sheet, such as a plurality of connected unit patterns form a three-dimensional macrostructure. In specific aspects, and the artificial support structure has a thickness between about 0.5 mm to about 1.5 mm, about 0.7 mm to about 1.3 mm, or about 0.9 mm to about 1.1 mm, in various embodiments. In some cases, the artificial support structure contains between 1 and 5 layers, and the layers may have thicknesses of between about 0.10 mm to about 0.3 mm, about 0.15 mm to about 0.25 mm, or about 0.18 mm to about 0.22 mm. In some embodiments, one or more filaments in the plurality of filaments have a diameter of less than about 550, 500, or 400 microns.

In various embodiments, columns or rows along which the plurality of unit patterns are arranged repeatedly are connected to allow the plurality of unit patterns to have intersection points with the plurality of unit patterns of the neighboring columns or rows. In certain embodiments, the plurality of unit patterns have the number of intersection points that is the same as the number of edges passing the intersection points. Four neighboring unit patterns are connected to have four intersection points with one another and have four edges passing the four intersection points, and a space surrounded with the four-unit patterns has the same or similar shape as or to each unit pattern, in certain cases. In particular embodiments, each unit pattern is an uppercase “I” of the English alphabet. In particular embodiments, short edges of the edges of the closed shape of each unit pattern have the same length as one another, and long edges thereof have the same length as one another, so that the space surrounded with the four-unit patterns has the same shape as each unit pattern, in various embodiments, and in the closed shape, the length ratio of a short edge to a long edge is 1:3, in certain cases. In the closed shape, a length ratio of each short edge to each long edge is 1:3, in specific embodiments. The plurality of unit patterns in the columns or rows along which the plurality of unit patterns is repeatedly arranged to have an angle of 45° or 135° with respect to the rows or columns, in various embodiments. In some embodiments, portions of the edges of the plurality of unit patterns are arranged regularly to form the edges of the artificial support.

In various embodiments, the artificial support structure has an ultimate tensile strength of between about 4 MPa to about 5 MPa, about 4.05 MPa to about 4.7 MPa, or about 4.12 MPa to about 4.50 MPa. In some embodiments, the artificial support structure has a modulus of elasticity of about 2.8 MPa to about 4.2 MPa, about 3.00 MPa to about 4.10 MPa, or about 3.06 Mpa to about 4.00 Mpa. In certain embodiments, the artificial support structure has a suture retention strength of about 20 N to about 26 N, about 21 N to about 25 N, or about 22.03 N to about 24.27 N. In particular embodiments, the artificial support structure has a burst strength of about 140 N to about 170 N, about 145 N to about 163 N, or about 147.14 N to about 161.26 N. In various embodiments, the artificial support structure has a tear resistance of about 18 N to about 26 N, about 19 N to about 25 N, or about 19.87 N to about 24.92 N.

Embodiments of the disclosure include a scaffold, comprising a patterned polymeric substrate having a coating of one or more extracellular matrix (ECM) materials thereon, wherein the pattern of the polymeric substrate comprises adjacent rows of a series of unit cell structures each generally in the shape of the letter “I”, the unit cell structures aligned in the rows of the patterned polymeric substrate in a perpendicularly alternating pattern of the unit cell structures. In certain embodiments, the unit cell structures of the series are further defined as comprising a pore shaped as a central line greater in length than two substantially equal-length lines each perpendicular to opposite ends of the central line, and wherein the alternating pattern is configured such that each of the ends of the central line of the pore are generally perpendicular to a central line of a pore of an adjacent unit cell structure. The scaffold is configured as one or more sheets, each comprising a first planar side and a second planar side, in some embodiments, and the scaffold comprises 1, 2, 3, 4, or 5 sheets, or comprises at least or no more than 1, 2, 3, 4, or 5 sheets, in particular aspects. In some embodiments, the multiple sheets are configured such that a planar side of one sheet is adjacent to a planar side of another sheet.

In some embodiments, the scaffold comprises one or more of a defined shape, such as generally a line, a curve, a circle, a square, a crescent, a triangle, a rectangle, an oval, a trapezoid, a bowl, or wherein the scaffold comprises markings for one of more of said defined shapes, in particular cases.

In various embodiments, the polymeric substrate comprises Polycaprolactone, Polydioxanone, or a combination thereof. The one or more ECM materials comprise a single type of collagen or a combination of one or more types of collagen, in specific cases, and in some cases the collagen is sourced from tendon, rat tail, bovine, porcine, or is recombinant. In specific embodiments, the combination of one or more types of collagen comprises Type I collagen and Type III collagen, and in some cases the collagen is telocollagen derived from bovine tendon, rat tail tendon, or is recombinant.

In particular embodiments, the scaffold includes a coating that is comprised on the first side of the sheet, the second side of the sheet, or both the first and second sides of the sheet. The coating fills the pore of multiple unit cell structures of the scaffold, fills the pore of the majority of unit cell structures of the scaffold, or fills the pore of substantially all unit cell structures of the scaffold, in specific embodiments. The coating does not fill the pore of the majority of unit cell structures of the scaffold or does not fill the pore of substantially all unit cell structures of the scaffold, in at least some cases. In specific embodiments, the thickness of the scaffold is not greater than 1 mm.

In various embodiments, a scaffold comprises one or more therapeutic agents, and the one or more therapeutic agents comprises one or more growth factors, one or more cytokines, one or more chemokines, one or more drugs, or a combination thereof, in some embodiments.

Embodiments of the disclosure include methods of producing any scaffold encompassed herein, the method comprising (a) three-dimensionally printing the patterned polymeric substrate; (b) applying the one or more ECM materials to the substrate; (c) subjecting the substrate to one or more crosslinkers; (d) optionally washing the substrate; and (e) subjecting the substrate to conditions less than 15° C. in temperature, optionally wherein the one or more ECM materials and the one or more crosslinkers are mixed together prior to applying to the substrate. The polymeric substrate is comprised of Polycaprolactone, Polydioxanone, or a combination thereof, in some cases. In specific embodiments, applying comprises submerging the substrate in a solution of the coating, laying the substrate on top of a solution of the coating, and/or spraying, dropping onto, and/or laying the coating onto the substrate. The applying may be performed for 1-24 hours, 5-24 hours, 5-20 hours, 8-20 hours, 8-15 hours, or 9-11 hours, as examples. In specific embodiments, one or more crosslinkers is selected from the group consisting of 1,4-Butanediol Diglycidyl Ether (BDDE), hexamethylene diisocyanate (HDMI), glutaraldehyde (GA), genipin, and a combination thereof. In certain cases, (c) is performed for about 1, 2, 3, 4, 5, 6, 7, 8, 9, 10, 11, 12, 13, 14, 15, 16, 17, 18, 19, 20, 21, 22, 23, or 24 hours. In embodiments, the washing occurs with water and (d) is performed for about 1, 2, 3, 4, 5, 6, 7, 8, 9, 10, 11, 12, 13, 14, 15, 16, 17, 18, 19, 20, 21, 22, 23, or 24 hours, in certain aspects. In some embodiments, (e) is performed for about 1, 2, 3, 4, 5, 6, 7, 8, 9, 10, 11, 12, 13, 14, 15, 16, 17, 18, 19, 20, 21, 22, 23, or 24 hours. In at least some cases following (e), the produced scaffold is subject to drying. The method may further comprise applying one or more therapeutic agents to the scaffold, in certain cases, such as present in the polymer, the coating, both the polymer and the coating, and/or are applied to at least part of the outside of the scaffold, in particular aspects. Degradation of the patterned polymeric substrate is adjustable based on the concentration of the crosslinker, in certain embodiments. The method may comprise washing the substrate after subjecting the substrate to one or more crosslinkers, in specific embodiments. The scaffold is produced in a defined shape, such as configured based on formation by a mandrel or is a pre-programmed macrostructure that is 3D printed, in particular embodiments.

Embodiments of the disclosure include methods of reinforcing soft tissue in an individual in need thereof, comprising applying an effective amount of any scaffold encompassed herein to one or more soft tissue sites of the individual. The soft tissue may comprise muscle, tendon, ligament, fascia, fat, skin, nerve, blood vessel, or a combination thereof, in particular embodiments, and in specific aspects soft tissue comprises an injury, a surgical site, a birth deformity, diseased tissue, or a combination thereof. The soft tissue is of the breast, stomach, abdomen, groin, leg, arm, hand, face, pelvis, uterus, vagina, penis, cervix, brain, nose, ear, eyelid, heart, kidney, liver, bladder, prostate, larynx, trachea, or a combination thereof, in particular embodiments. In specific cases, the soft tissue comprises breast tissue for breast reconstruction, breast reduction, or breast enlargement or the soft tissue comprises a hernia. The applying comprises affixing the scaffold to the soft tissue of the individual and/or tissue adjacent to the soft tissue of the individual, in particular embodiments. The affixing is further defined as suturing, stapling, or using surgical glue to affix the scaffold to the soft tissue of the individual and/or tissue adjacent to the soft tissue of the individual, in some cases, and the suturing is purse-string suturing, continuous suturing, interrupted suturing, buried suturing, deep suturing, or subcutaneous suturing, in certain embodiments. The suture of the suturing is absorbable or is nonabsorbable.

Embodiments of the disclosure encompass a pliable sheet, comprising one or more biodegradable polymers and one or more ECM materials, said sheet comprising a plurality of patterned unit cell structures aligned in adjacent rows of a series of perpendicularly alternating unit cell structures each generally comprising a pore shaped having a central line greater in length than two substantially equal-length lines each perpendicular to opposite ends of the central line, wherein the alternating pattern is configured such that each of the ends of the central line of the pore are generally perpendicular to a central line of a pore of an adjacent unit cell structure. The unit cell structures are comprised of one or more biodegradable polymers, in certain embodiments, and the sheet comprises a coating of one or more ECM materials. In specific embodiments, the pliable sheet is further defined as the unit cell structures comprising a coating of one or more ECM materials. The pores are filled with the coating, in some cases, and the one or more ECM materials comprise Type I collagen. The sheet is housed in suitable packaging, in certain cases and is sterile in particular embodiments.

It is contemplated that any embodiment discussed in this specification can be implemented with respect to any method or composition of the disclosure, and vice versa, and different embodiments may be combined. Furthermore, compositions of the disclosure can be used to achieve methods of the disclosure. The claims originally filed are contemplated to cover claims that are multiply dependent on any filed claim or combination of filed claims. The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Other objects, features and advantages of the present invention will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The disclosure may be better understood by reference to one or more of these drawings in combination with the detailed description presented herein.

FIG. 1A. An illustration of an embodiment of multiple unit patterns.

FIG. 1B. A close view of an example of multiple unit patterns configured in a 3D-printed scaffold.

FIG. 1C. A top view of an example of a scaffold prior to deposition of an extracellular matrix coating.

FIG. 1D. One view of an example of a scaffold prior to deposition of an extracellular matrix coating.

FIG. 1E. A side view of an example of a scaffold prior to deposition of an extracellular matrix coating.

FIG. 1F. A side view of an example of a scaffold to demonstrate scale.

FIG. 2. Microscopic images of polyglactin 910 mesh* and PCL Soft Tissue Reinforcement Scaffold (STRS)** at 0 and 6 weeks (*: longitudinal direction, **: pattern P).

FIG. 3A. Ultimate Tensile Strength (UTS) of polyglactin 910 mesh over Time (longitudinal direction).

FIG. 3B. Young's Modulus (YM) of polyglactin 910 mesh over Time (longitudinal direction).

FIG. 4A. UTS of PCL STRS over Time.

FIG. 4B. YM of PCL STRS over Time.

FIG. 5. Result of Enzymatic Degradation Test (BDDE x %: collagen matrix using x % BDDE).

FIG. 6. UTS graph comparing a STRS of the disclosure to TnR Mesh and an ADM control (MegaDerm).

FIG. 7. Comparison of cell infiltration of ADM vs. STRS.

FIG. 8. Graphs showing control of degradation upon adjustment of crosslinker concentration.

FIG. 9. One example of a Manufacturing Process of STRS.

FIG. 10. One example of Freeze-Drying conditions for one or more actions in STRS Manufacturing Process. The term “9999” in SVP refers to maintaining a constant temperature (different from room temperature) until the sample is recovered.

FIG. 11. UTS graph comparing different STRS manufactured with either PCL or PDO at specific line widths, in comparison to MegaDerm.

FIG. 12. Bubble Chart of STRS and MegaDerm.

### DETAILED DESCRIPTION

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular.

This specification describes exemplary embodiments and applications for the disclosure. The disclosure, however, is not limited to these exemplary embodiments and applications or to the manner in which the exemplary embodiments and applications operate or are described herein. Other embodiments, features, objects, and advantages of the present teachings will be apparent from the description and accompanying drawings, and from the claims. Moreover, the figures may show simplified or partial views, and the dimensions of elements in the figures may be exaggerated or otherwise not in proportion. Section divisions in the specification are for ease of review only and do not limit any combination of elements discussed.

### I. Examples of Definitions

The use of the word “a” or “an” when used in conjunction with the term “comprising” may mean “one,” but it is also consistent with the meaning of “one or more,” “at least one,” and “one or more than one.”

Throughout this application, the term “about” is used to indicate that a value includes the inherent variation of error for the measurement or quantitation method.

It is contemplated that any method or composition described herein can be implemented with respect to any other method or composition described herein and that different embodiments may be combined.

As used herein, “bioink” may denote any bioactive, bioprintable, naturally or artificially derived material that can be deposited as filaments, fibers, fibrils, droplets, gels/hydrogels, or slurry during an additive manufacturing process and may be utilized to mimic an extracellular matrix environment to support the adhesion, proliferation, and differentiation of living cells. In specific cases, the bioink may provide material to facilitate soft tissue reinforcement.

As used herein, “extracellular”, as used in reference to, for example, “extracellular material”, “extracellular structure”, “extracellular matrix”, “extracellular construct”, and “extracellular component”, may denote the characteristic of existing outside the cell and can refer to a synthetic or natural material. Examples of materials that are extracellular include synthetic and natural polymers; metabolites; ions; various proteins and non-protein substances (e.g. DNA, RNA, lipids, microbial products, etc.) such as collagens, proteoglycans, hormones, growth factors, cytokines, chemokines; various enzymes including, for example, digestive enzymes (e.g., Trypsin and Pepsin), extracellular proteinases (e.g., matrix metalloproteinases, a disintegrin and metalloproteinase with thrombospondin motifs (ADAMTSs), Cathepsins) and antioxidant enzymes (e.g., extracellular superoxide dismutase); proteolytic products; extracellular matrix proteins (such as elastin, glycosaminoglycans (GAGs), laminin, fibronectin, etc.), selected cell populations, small molecules and small molecule inhibitors, antibiotics, antimicrobials, nanoparticles, mesoporous silica, silk fibroin, enzymatic degradation sites; anti-fibrotic agents such as anti-transforming growth factor beta (anti-TGF-β) and anti-tumor necrosis factor alpha (anti-TNF-α); pro-angiogenic agents such as vascular endothelial growth factor (VEGF) and placental growth factor (PlGF); and factors affecting adipogenesis and proliferation such as insulin-like growth factor 1 (IGF-1) and Dexamethasone.

The term “pore” as used herein may refer to an opening in a scaffold. The pore may or may not be of a specific shape. The shape of the pore may not be circular or square but may be generally in the shape of the letter “I.” The pore may comprise the shape of a central line having two shorter perpendicular lines at opposing ends of the central line.

As used herein, the term “resorbable” may refer to the capacity of a device to undergo biodegradation (chemical breakdown by biological agent) and the degradation products removed by cellular activity in a biological environment.

As used herein, “scaffold” may denote a biocompatible and bioresorbable structure used in tissue engineering that is capable of being implanted in the body in order to provide support and/or promote cell adhesion and tissue regeneration, such as for diseased tissue or wound repair. The scaffold may comprise repetitions of defined unit patterns in adjacent rows. A scaffold can be used, for example, in the areas of soft tissue, including for regeneration and/or reinforcement of cartilage, skin, organ, muscle, tendon, ligament, fascia, fat, skin, nerve, blood vessel, or a combination thereof. The scaffold may be utilized in soft tissue of the breast, stomach, abdomen, groin, leg, arm, hand, face, pelvis, uterus, vagina, penis, cervix, brain, nose, ear, eyelid, heart, kidney, liver, bladder, prostate, larynx, trachea, or a combination thereof. The term “artificial support structure” may be used herein interchangeably with the term “scaffold.”

As used herein, the term “unit pattern” and “unit cell structure” may be used interchangeably and may refer to a recurring shape having a defined outline that borders a defined pore. The outline and the pore may be generally of the same shape. The unit cell structure may be a structure having the general shape of the letter “I.”

### II. Scaffold and Uses Thereof

There are a variety of material options to provide tissue reinforcement or fill-in resected tissue to reconstruct the native shape of soft tissue, including in areas where the resected tissue was taken. Examples of materials include acellular dermal matrix (ADM) bioscaffolds obtained from decellularized human, bovine, or porcine dermis; fat or other grafted tissue fillers; synthetic polymer-based scaffolds; or a combination thereof. However, certain materials have a number of shortcomings, including for example causing deleterious immunogenic responses in the recipient (such as with ADM); lacking support, protection, and reinforcement to the dermis layer to be able to maintain shape or support; and lacking the ability to provide robust cellular infiltration/remodeling response post-implantation, as examples.

Certain bioscaffolds may fail to provide compositions of extracellular matrix proteins that facilitate cell ingrowth into the bioscaffolds, Such scaffolds may not therefore enhance tissue regeneration, that can assist with patient recovery. In certain embodiments, he compositions of the present disclosure utilize a combination of a 3D-printed polymer scaffold and animal-derived collagen in a certain pattern as an improvement over other synthetic scaffolds by providing native mechanical tissue properties that they lack.

Disclosed herein are compositions, methods, and materials that relate to an implantable acellular scaffold that contains native extracellular matrix (ECM) proteins that can elicit a robust regenerative response while minimizing undesirable immunogenic responses from the host. Such a scaffold can provide support to a wound or fill a void post-surgery in addition to enhancing host cell ingrowth, regeneration, and repair.

The present disclosure concerns three-dimensional printing of scaffolds of varying macroshape based on bioprinting configurations, including at least flat sheets and/or 3D scaffold structures. The scaffold composition can provide cushioning and structural support for other tissues, supplemental support, protection, reinforcement, and covering within any soft tissue, including muscle, tendon, ligament, fascia, fat, skin, nerve, blood vessel, or a combination thereof. The soft tissue may be located anywhere in the body, and the scaffold can be configured to stimulate host cell remodeling.

The scaffold may be biodegradable or readsorbable. It can be utilized to support, repair, elevate, and reinforce deficiencies where weakness or void(s) exist in soft tissue (or where both weakness and void(s) exist) that requires the addition of the material. The scaffold can facilitate repair of defects that require the addition of a reinforcing, regenerative, and/or bridging material, e.g., to obtain a desired surgical result.

In various embodiments, the scaffold composition can minimize host immunogenic response, given that the scaffold may comprise user-selected or desired components. Unlike current acellular dermal matrix (ADM) products, which can trigger a “graft vs. host response,” the various scaffold compositions encompassed herein allow for the control of components and allow for standardization of clinical outcomes within and among patients. The scaffold provides increased support and reinforcement compared to other available products, and the scaffold can provide controlled, enhanced elasticity and tensility in relation to currently available products in the art. The utilization of 3D printing and this particular pattern/material combination have the ability to recapitulate native mechanical tissue properties that other synthetic scaffolds cannot.

The present disclosure relates to printable scaffold compositions that mimic acellular matrix products yet have unique printability into 2D and 3D shapes and the ability to support tissue and/or organ growth. The scaffold compositions described herein are improved at least because they minimize concerns over donor availability, provide for reproducibility, alleviate increasing costs, eliminate concerns over tissue quality, variability and contamination potential, and provide a minimally immunogenic product. The scaffold (including the unit cell pattern and material) can also be tailored to impart different mechanical properties as required by the end user in response to clinical need, which cannot be done with other synthetic scaffolds or allograft/xenograft tissue. The scaffold may be configured to be utilized permanently in the body.

A scaffold encompassed herein can provide the requisite porosity to allow cellular infiltration and provide a large enough niche for cells to attach, ultimately directing cell fate towards a remodeling/regenerative phenotype. Furthermore, from a mechanical/structural perspective, the scaffold comprises a plurality of particular unit cell structures, and the arrangement of the unit cell structures within the scaffold structure provides the appropriate mechanical strength and elasticity for the scaffold to be physiologically relevant as well as useful as a supportive matrix. These features can be provided by the scaffold structure using, for example, an extracellular material composition as disclosed herein, such as one comprising Collagen I, thereby providing the necessary structural integrity and healing properties.

The unit cell structure configuration of the scaffold imparts to the scaffold specific physical properties, and the construct can have consistent surface topography throughout with an engineered microarchitecture (controlling properties such as, for example, porosity, fiber diameter, spacing, height of matrix, fiber orientation, etc.) that provides the appropriate scaffold for a robust wound healing, regenerative, infiltrative, and/or remodeling response.

Moreover, the construct can provide, for example, cushioning and structural and mechanical support for other tissues, supplemental support, protection, reinforcement and covering within the soft tissue while stimulating host cell remodeling.

A. Scaffold Materials

A scaffold composition is provided that may comprise one or more biodegradable polymers, one or more extracellular matrix materials, and optionally other components, such as one or more therapeutic agent(s). The unique combination of biodegradable polymers and extracellular matrix materials together provide the appropriate support matrix for remodeling and provides a fertile environment for cell infiltration. The combination of the polymeric and biologic construct materials also provides a robust scaffold for the addition of other molecular moieties, in various embodiments.

The scaffold may comprise natural and/or synthetic polymers including any polymer(s) that provides mechanical stability, and it may have a consistent degradation profile that allows greater predictability for individuals receiving the scaffold. The scaffold composition may comprise any suitable natural or synthetic polymer or combinations or blends thereof. The synthetic polymer may be biodegradable and can include, for example, polycaprolactone (PCL), Poly(p-dioxanone) (PDO), a combination thereof, or any other type of polymer.

The deposition of the polymer of the unit cell structure can be performed, for example, by a bioprinter using components such as, for example, a nozzle or syringe. These components can be, for example, pneumatically, piston, or screw-driven. Pneumatic driven syringes, for example, can deposit liquefied polymer in sequential layers to generate the construct, which will ultimately be cross-linked.

The synthetic polymer may be deposited by a 3D printer as a bioink. The bioink may or may not comprise bioactive molecules. As compared to traditional polymer-based compositions, certain bioinks comprising bioactive molecules in addition to polymers may have to be deposited under milder conditions relative to polymer-based compositions. This can be due to the relatively more delicate nature of bioink structures (e.g., higher water content, non-crystalline structure, etc.). As such, bioprinting process parameters such as printing pressure or nozzle/syringe diameter can be considered in reducing the shear stress on some bioinks to prevent damaged or lysed cells, which can affect cell viability in the bioinks. Other parameters that may be considered, and correspondingly controlled include, for example, printing temperature (e.g., lower temperature than polymer-based compositions), uniformity in diameter of the filaments that make up the unit cell, angles at the interaction of filaments, bleeding of filaments together at intersects, and maintenance of shape fidelity after printing but before cross-linking with polymer-based compositions. In any event, the polymer may be deposited as, for example, droplets or streams utilizing defined process parameters to ensure scaffold manufacturing while maintaining structural integrity of the deposited compositions.

Each unit cell of the scaffold structure can comprise a polymer or blend of polymers and can comprise an ECM material coating that may or may not comprise collagen. It should be appreciated that the extracellular material of the scaffold structure, which can include PCL and/or PDO and/or another dissolvable or liquefiable polymer, can aid in providing the structural integrity and function of the scaffold structure as well as the unit cells that make up the structure.

The extracellular matrix component of the scaffold may comprise one or more of collagen (e.g., collagen 1 (Col-1), and optionally other types of collagen), extracellular matrix proteins (e.g., laminin, fibronectin, elastin, glycosaminoglycans, or combinations thereof), growth factors, cytokines, selected cell populations, small molecules, small molecule inhibitors, antibiotics, antimicrobials, nanoparticles, mesoporous silica, silk fibroin, and enzymatic degradation sites.

The scaffold can comprise an ECM material coating instead of a collagen coating, a collagen coating, or a combined collagen/ECM material coating, in certain embodiments.

B. Scaffold Shape and Structure

The disclosure is generally directed towards scaffold structures that can include unit cells. More specifically, there is a need for porous extracellular structures and/or scaffold structures that have a porous construct suited to promote cellular infiltration, tissue regeneration, and minimize risk of adverse immune response in and/or pathogenic contamination to the patient.

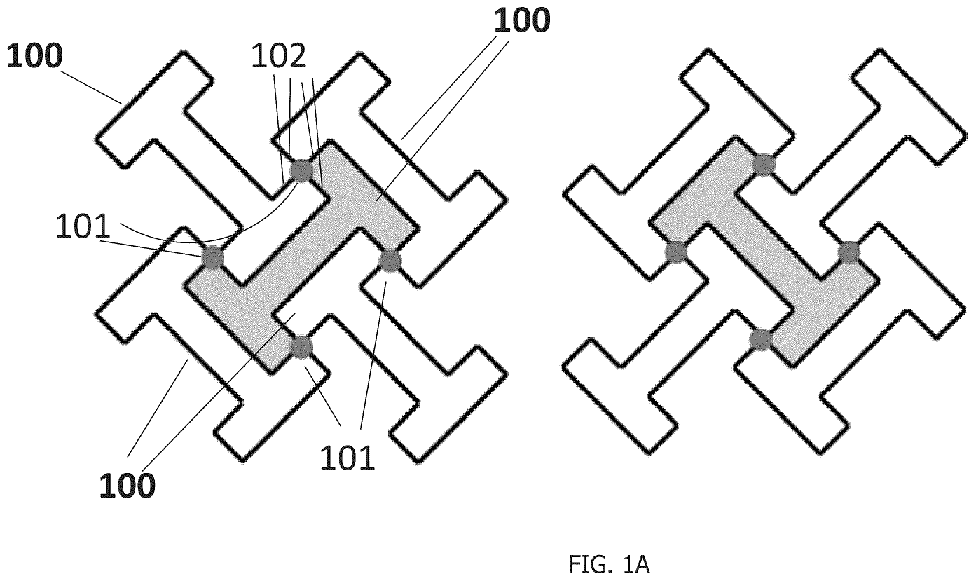

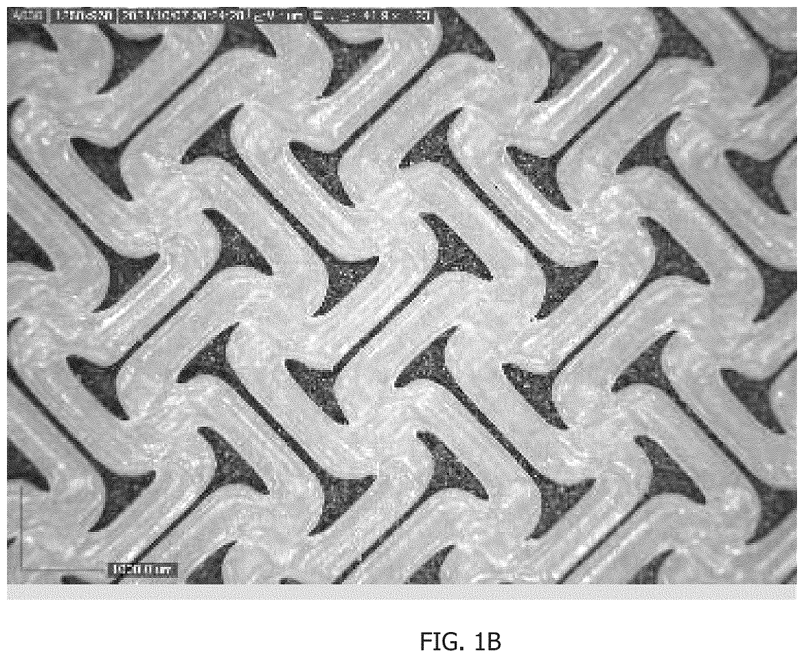

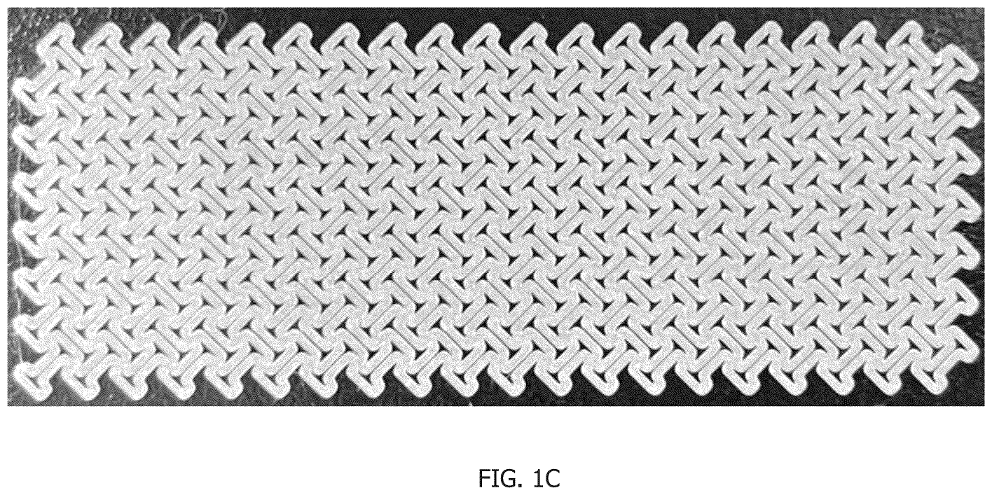

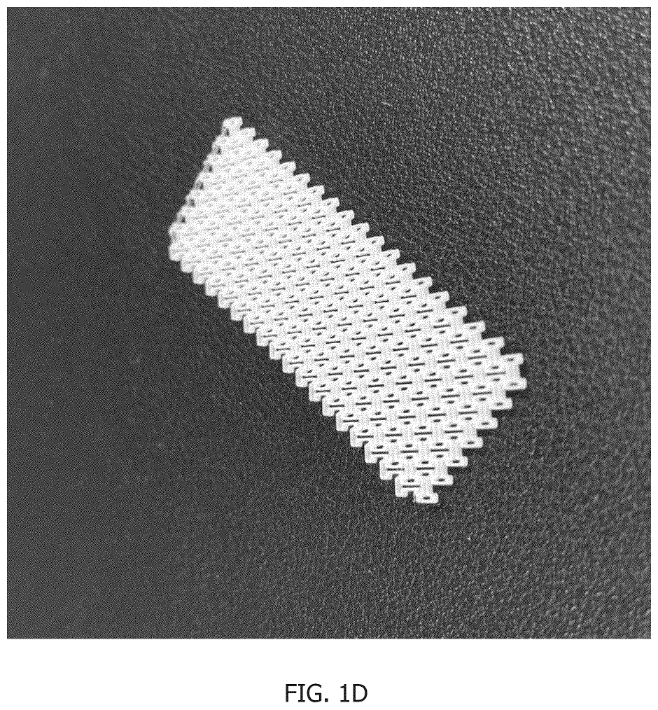

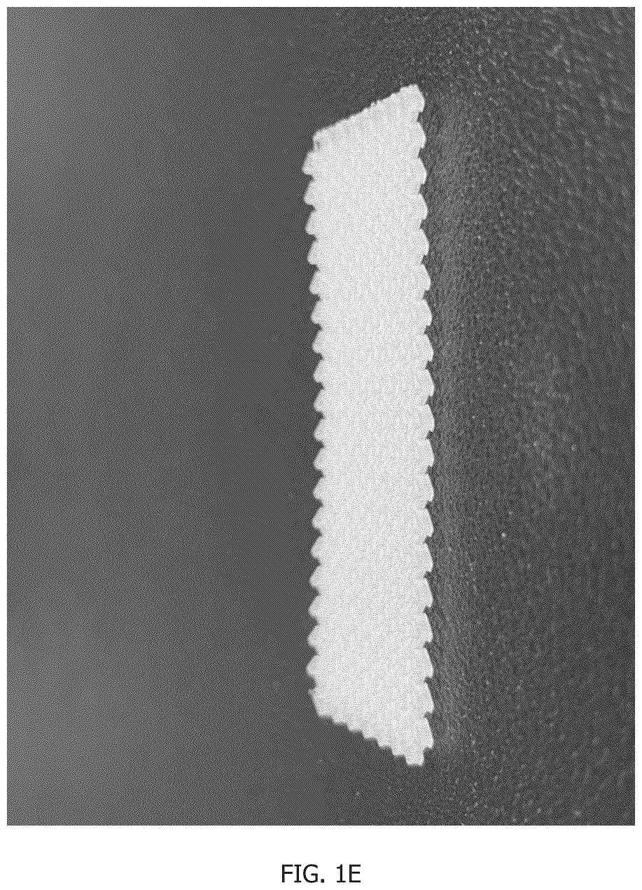

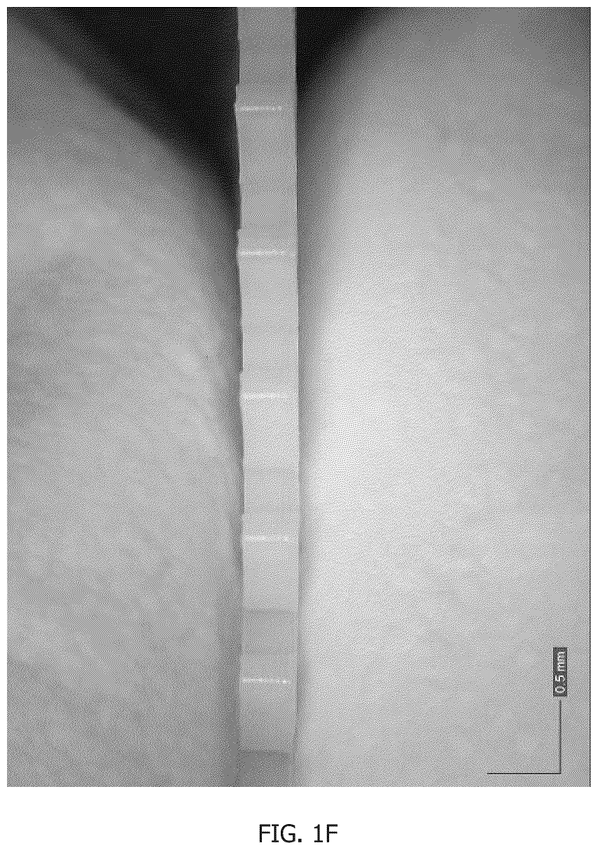

The scaffold of the disclosure comprises a plurality of unit parts of a defined shape and structure. The scaffold may be configured of a plurality of unit cell structures arranged in or constituting a constant or definite pattern. The scaffold may be comprised of a plurality of unit patterns, which may also be referred to as a unit cell structure, each unit pattern comprising a plurality of filaments, arranged continuously, symmetrically, and regularly thereon. Each unit pattern may be constituted of edges or outlines of a closed shape that then forms a pore at the inside. The plurality of unit patterns in the scaffold may be connected to have intersection points with one another and the number of intersection points may be the same as the number of edges passing the intersection points (FIG. 1A). As represented in FIG. 1A, for unit pattern 100, the number of intersection points 101 is the same as the number of edges/lines 102 at a particular intersection point 101.

The scaffold may be considered an artificial support structure comprising a plurality of unit patterns that each comprise a plurality of filaments that are arranged repeatedly to establish columns or rows symmetrical to one another. In such cases, each unit pattern may be comprised of edges of a closed shape that produce a pore at the inside, and the columns or rows along which the plurality of unit patterns may be arranged repeatedly have an Eulerian trail.

A scaffold can comprise a patterned polymeric substrate having a coating of one or more ECM materials, and the pattern of the polymeric substrate may comprise adjacent rows of a series of unit cell structures each generally in the shape of the letter “I.” In such cases, the unit cell structures may be aligned in the rows of the patterned polymeric substrate in a perpendicularly alternating pattern of the unit cell structures. The unit cell structures of the series may be further defined as comprising a pore shaped as a central line greater in length than two substantially equal-length lines each perpendicular to opposite ends of the central line, and the alternating pattern within the scaffold may be configured so that each of the ends of the central line of the pore are generally perpendicular to a central line of a pore of an adjacent unit cell structure.

FIG. 1B shows a close-up image of an example of a bioprinted scaffold prior to deposit of the one or more ECM materials thereon. The filaments making up the outline of the I-shaped unit cell structure may be deposited by a 3D printer using a particular line width. FIGS. 1C and 1D provide different angles of images of the entirety of one sheet of scaffold of the same unit cell structure configurations as in FIG. 1B. A given row or column in a scaffold may or may not be generally diagonal with respect to the edges of the scaffold structure. FIGS. 1E and 1F provide different angles of images of the side of one sheet of scaffold. The thickness of the scaffold may be pre-determined by the deposition of the 3D-printed polymer.

Degradation of the scaffold composition may occur over time following surgical implantation. The degradation of the scaffold composition may occur partially or fully over, or over about, 6, 7, 8, 9, 10, 11, or 12 months. The degradation of the scaffold composition may occur partially or fully over, or over about, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 months. The degradation of the scaffold composition may occur partially or fully over, or over about, 1, 2, 3, 4, 5, 6, 7, 8, 9, 10, or more years. The degradation of the scaffold composition may occur over, or over about, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 years. Host cell integration into the porous scaffold can proceed throughout the degradation process, such that infiltrating cells will degrade the polymer and secrete their own extracellular matrix in an effort to regenerate that tissue. The scaffold composition degradation profile is definable and predictable across manufactured lots, which is an improvement over the currently available acellular dermal matrix constructs in the art.

The plurality of connected unit cells in the scaffold structure can form a substantially planar sheet.

The artificial support structure or scaffold may comprise particular dimensions and/or mechanical properties that can impart native mechanical tissue properties. The compositions may have a particular thickness, such as between about 0.5 mm to about 1.5 mm, about 0.7 mm to about 1.3 mm, or about 0.9 mm to about 1.1 mm. In some cases, a layer for the compositions may have a thickness between about 0.10 mm to about 0.3 mm, about 0.15 mm to about 0.25 mm, or about 0.18 mm to about 0.22 mm. In cases wherein multiple layers are utilized, the separate layers may or may not have the same thickness. For the filaments of the compositions, they may have a diameter of less than about 550, about 500, or about 400 microns.

The artificial support structure or scaffold may have an ultimate tensile strength of between about 4 MPa to about 5 MPa, about 4.05 MPa to about 4.7 MPa, or about 4.12 MPa to about 4.50 MPa. With respect to modulus of elasticity, any composition may have a modulus of about 2.8 MPa to about 4.2 MPa, about 3.00 MPa to about 4.10 MPa, or about 3.06 Mpa to about 4.00 Mpa. The artificial support structure or scaffold may be affixed to soft tissue by any manner, including with sutures, and a suture retention strength may be about 20 N to about 26 N, about 21 N to about 25 N, or about 22.03 N to about 24.27 N. For additional mechanical properties, the artificial support structure may have a burst strength of about 140 N to about 170 N, about 145 N to about 163 N, or about 147.14 N to about 161.26 N and/or may have a tear resistance of about 18 N to about 26 N, about 19 N to about 25 N, or about 19.87 N to about 24.92 N.

The scaffold may be printed to a desired shape ahead of its need, such as being placed into storage or to be sold commercially. In such cases, the surface of the scaffold may be 2D and the printed scaffold may be 3D and may be a pre-fabricated shape, or it may be customized to a particular patient's need, such as a particular wound or void of the patient that requires personal adaptation. The shape of the scaffold may be based on a mandrel or pre-programmed macrostructure that would be 3D printed. The design of the shape of the scaffold may be produced using Computer-Aided Manufacturing (CAM) software prior to manufacture. The scaffold may be in the shape of a sheet, such as a square or rectangle, for example, or it may be in the shape of a bowl or other 3D ultrastructure that matches patient anatomy. The sheet may be cut to a specified size and/or shape based on a patient's need. The scaffold may be stored or commercially sold in any form, including sheet form or as a printed 3D structure.

C. Additional Scaffold Components

The scaffold of the disclosure may be manufactured such that the structure comprises one or more therapeutic or other agents. The agent(s) may be eluted from the scaffold, as part of a coating on the scaffold, a combination thereof, and so on. The agent(s) may be useful as a therapeutic for the tissue adjacent to the scaffold, near the scaffold, and so on, such as being wound-healing, diseased tissue-healing, and so forth. The scaffold may be configured such that being resorbable over a period of time (such as noted above) allows for delivery of the agent or agents over the period of time. The agent(s) may be utilized for wound healing, scar prevention, fibrosis prevention, and/or long-term treatment for chronic or recurring medical conditions. The release of the agent from the scaffold may be a modified-release, such as immediate-release, sustained-release, delayed-release, or a controlled-release whereby the rate of agent release may be controlled.

The agent(s) may be provided on one or more exterior surfaces of the scaffold and/or is incorporated within the scaffold. The agent(s) can be mixed with a precursor polymer solution and become incorporated within the polymer matrix during fabrication with release occurring upon degradation of the polymer. The agent can be incorporated within a device by placing the device in a solution of the agent and allowing the agent to adsorb to the surface of the device with release controlled by desorption rates. The agent can be covalently grafted to functional groups on device polymeric chains. The covalent-attachment groups can be selected to be labile groups, e.g., ester or thio-β ester groups, that will become cleaved and release the agent in a physiological environment. The agent can be incorporated into the ECM coating.

The time period in which an agent elutes from the scaffold is substantially the same time period of use of the scaffold, including as at least part of the scaffold is resorbed by the body. Substantially all of the agent may elute from the scaffold prior to partial or complete resorption by the body; in such cases, a sufficient amount of the agent is utilized in order to provide sufficient healing at the site of use.

The one or more therapeutic agents may be of any kind that are suitable for an individual receiving the composition for any purpose. The agent(s) to which the composition is associated may be tailored to the therapeutic need of the individual. For example, the individual may be in need of a specific agent or agents as part of the composition based on a medical condition of the individual, and the composition is manufactured comprising the agent(s) accordingly. The individual may be in need of a specific agent or agents as part of the composition based on a medical condition of the individual, and the composition comprising the desired agent(s) is obtained already manufactured, such as upon storage, commercially, and so forth.

The therapeutic agent may be a bioactive molecule, growth factor, cytokine, chemokine, drug, hormone, antibiotic, pain reliever, hemorrheologic, vasoconstrictive, anti-inflammatory, anti-fibrotic, wound-healing agent, radioprotective material, anti-fungal, contraceptive, or any combination thereof. In cases wherein the therapeutic agent is a growth factor, the growth factor may be Epidermal growth factor, keratinocyte growth factor, transforming growth factors including TGF-α, TGF-β1, TGF-β2, vascular endothelial growth factor, platelet-derived growth factor, a blocking factor, scavenger, antagonist, differentiation factor, or an agent that binds a particular promoter. In cases wherein the therapeutic agent is a cytokine, the cytokine may be Interleukin-2 (IL-2), IL-7, IL-15, or a combination thereof. In cases wherein the therapeutic agent is a chemokine, the chemokine may be any one or more of the chemokines in the following subclasses: CXCL-, CCL-, CX3-, XCL-. Specific examples of chemokines include CXCL1, CXCL2, CXCL3, CXCL4, CXCL4L1, CXCL5, CXCL6, CXCL7, CXCL8, CXCL9, CXCL10, CXCL11, CXCL12, CXCL13, CXCL14, CXCL15, CXCL16, CXCL17, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CX3CL1, XCL1, XCL2, or a combination thereof. In cases wherein the therapeutic agent is a drug, the drug may be a small molecule, antibody, nucleic acid, polypeptide, carbohydrate, or combination thereof. In specific cases wherein the therapeutic agent is a drug, the drug may be an analgesic, anesthetic, antibacterial, antifungal, antiviral, anti-inflammatory, hormone, vitamin, mineral, immunological agent, or a mixture thereof.

The agent may be a hormone, antibiotic, pain reliever, hemorrheologic, vasoconstrictive, anti-inflammatory, anti-fibrotic, wound-healing agent, radioprotective material, anti-fungal, contraceptive, or any combination thereof. In specific cases, the agent is a drug, such as AMD3100, tacrolimus, 2-octyl cyanoacrylate, Alevicyn, Artiss, Becaplermin, Betaine/polyhexanide, Cadexomer iodine. Collagenase, Dermabond, Eletone cream, Episalvan, Evicel, Fibrin sealant, Filsuvez, Hypochlorous acid topical, Lodosorb, NexoBrid, Oleogel-S10, Petrolatum & mineral oil topical, Prontosan, Proteolytic enzyme, Regranex gel, Santyl, TachoSil, Tisseel VH, Tropazone, or a combination thereof. When the agent is a hormone, the hormone may be estrogen.

If desired, the scaffold composition may comprise one or more growth factors. The growth factors may be any one or combination of e.g., GM-CSF, NGF, SCF, TGF-0, EGF, VEGF and others.

The scaffold composition may further comprise one or more cytokines. The cytokines may be any one or combination of e.g., IL-1, IL-4, IL-5, IL-6, IL-9, IL-13, IL-18, IL-25, IFN-α, IFN-β, and others.

If desired, the scaffold may further comprise one or more antibiotics. Suitable antibiotics include a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics and the like.

The scaffold composition may further comprise one or more antimicrobials, including antibacterials, antifungals (e.g., polyene antifungals, such as amphotericin B; triazole antifungals, such as itraconazole, ketoconazole, fluconazole, voriconazole, clotrimazole, Isavuconazole, miconazole and posaconazole; echinocandin antifungals, such as caspofungin, micafungin, and anidulafungin, orotomide antifungals, such as F901318, which inhibits dihydroorotate dehydrogenase), antivirals (e.g., oseltamivir, zanamavir, amantidine, rimantadine, ribavirin, gancyclovir, valgancyclovir, foscavir, Cytomegalovirus Immune Globulin, pleconaril, rupintrivir, palivizumab, motavizumab, cytarabine, docosanol, denotivir, cidofovir, and acyclovir), antiparasitics or combinations thereof.

The scaffold composition may further comprise one or more pro-angiogenic (e.g., VEGF, PlGF) bioactive molecules to promote vascularization in patients with or without compromised vascularity.

The scaffold composition may further comprise one or more anti-fibrotic molecules (anti-TGFβ, anti-TNF-α), such as to reduce fibrosis in patients at the implant site.

The scaffold composition may further comprise one or more factors affecting adipogenesis and proliferation (e.g., IGF-1, Dexamethasone) to promote grafted adipose cell growth.

### III. Methods of Use

The disclosure includes scaffold compositions and methods for tissue care of any kind, including treatment for a wound, diseased tissue, or medical condition of any kind that impacts a soft tissue location in vivo. Although the scaffold can be utilized for anyone that is in need, the scaffold may be for a mammal, including a human, and the human may comprise soft tissue in need of the scaffold. The individual in need may be an individual having soft tissue that is wounded, diseased, in need of post-operational treatment, in need of perioperative treatment in need of gender reassignment, and/or in which a medical condition has resulted in need of treatment of the soft tissue. A wound may be internal or external and may be from trauma, disease, laceration, impaired circulation, neuropathy, a surgical procedure, and so forth. Operational or post-operational treatment may require use of the scaffold, such as from obstetric surgery or procedure, male-to-female transgender surgery, breast reconstruction, hernia repair, and so forth. The scaffold may be used for reconstructive or cosmetic purposes, in some cases.

The scaffold may be obtained off-the-shelf or may be manufactured upon need of the scaffold. In either case, the type of need and/or size of the individual receiving the scaffold may be taken into account prior to manufacture or selection of the device. The individual receiving the scaffold may be pediatric (up to 12 years of age) or adolescent (12-18 yrs of age) or an adult. Part or all of the scaffold may be designed or configured to be utilized for permanent use, although part of the scaffold may be designed or configured to disappear or dissolve over time, including being resorbed by the body.

The degradation of part or all of the scaffold may or may not conform to the timing of the healing. For example, the tissue may be healed prior to resorption of part of the scaffold, or the tissue may not be healed completely prior to resorption of part of the scaffold and an additional treatment may then be utilized if needed.

Specific examples of uses of the device include those for operative, post-operative, and/or post-radiation settings, such as to facilitate wound healing or prevent scarring or occlusion. In any event, particular examples include at least treatment after radiation and/or surgery, fibrosis of any kind or degree and including treatment or prevention, post-operational treatment, physical damage or injury, shortening and/or tightening of tissue from surgery and/or radiation, cervical incompetence, vaginal/uterine prolapse, or a combination thereof.

The disclosure provides a method of reinforcing soft tissue in an individual in need thereof that may comprise applying an effective amount of the scaffold to one or more soft tissue sites of the individual. The soft tissue can comprise muscle, tendon, ligament, fascia, fat, skin, nerve, blood vessel, or a combination thereof, as examples. The soft tissue may comprise an injury, a surgical site, a birth deformity, diseased tissue, or a combination thereof. In particular, the soft tissue may be of the breast, stomach, abdomen, groin, leg, arm, hand, face, pelvis, uterus, vagina, penis, cervix, brain, nose, ear, eyelid, heart, kidney, liver, bladder, prostate, larynx, trachea, or a combination thereof. The soft tissue may comprise breast tissue for breast reconstruction, breast reduction, or breast enlargement.

The scaffold may be applied to the soft tissue of the individual in any suitable manner. The application may comprise affixing the scaffold to the soft tissue of the individual and/or affixing the scaffold to tissue adjacent to the soft tissue of the individual. The affixing may comprise suturing, stapling, and/or using surgical glue to affix the scaffold to the soft tissue of the individual and/or tissue adjacent to the soft tissue of the individual. The wound or void (or both in the same tissue) being repaired may dictate the type of affixing and, if suturing is occurring, the suturing may be purse-string suturing, continuous suturing, interrupted suturing, buried suturing, deep suturing, or subcutaneous suturing, as examples. The sutures for the suturing may be absorbable or may be nonabsorbable.

### IV. Methods of Manufacture

The manufacture of the scaffold may comprise a series of ordered actions, at least in part. The method may generally comprise production of the scaffold by a bioprinter (the synthetic polymer) that occurs separately from preparation of the one or more ECM materials (the natural polymer, in at least some cases, including those that utilize collagen) that is then combined with the crosslinker. Following combination of the synthetic and natural polymers, the combination is subject to an effective amount of crosslinker, followed by washing (optional) and then freeze drying.

A method of producing the scaffold encompassed by the disclosure may include (a) three-dimensionally printing the scaffold as the patterned polymeric substrate; (b) applying one or more ECM materials to the substrate; (c) subjecting the substrate to one or more crosslinkers; (d) optionally washing the substrate; and (e) subjecting the substrate to conditions freeze-drying, such as a temperature less than 15° C., optionally wherein the one or more ECM materials and the one or more crosslinkers are mixed together prior to applying to the substrate.

The scaffold may be bioprinted using a suitable synthetic, biodegradable polymer, such as PCL, PDO, or a combination thereof. The bioprinting of the polymer may follow a particular pattern to produce the unit cell structures generally formed as a filament outline of a shape of an “I” having a pore similarly shaped. The line width of the filament may be optimized and may be about 400 μm for PCL and about 500 μm for PDO. As a separate action, the one or more ECM materials (again, a natural polymer in some cases) is prepared or obtained and may be mixed with an effective amount of crosslinker (one or more crosslinkers is selected from the group consisting of 1,4-Butanediol Diglycidyl Ether (BDDE), hexamethylene diisocyanate (HDMI), glutaraldehyde (GA), genipin, and a combination thereof) to produce a coating for the scaffold. Following this, the coating mixture to be imparted onto the scaffold is applied to the scaffold in any suitable manner, such as submerging the scaffold in a solution of the coating, laying the scaffold on top of a solution of the coating, spraying the coating onto the scaffold, dropping the scaffold onto the coating, or laying the coating onto the substrate.

Applying of the one or more ECM materials and/or the one or more crosslinkers may be performed for any suitable amount of time, such as 1, 2, 3, 4, 5, 6, 7, 8, 9, 10, 11, 12, 13, 14, 15, 16, 17, 18, 19, 20, 21, 22, 23, or 24 hours. Applying may be performed for 1-24, 1-18, 1-12, 1-10, 1-8, 1-6, 1-2, 2-24, 2-18, 2-16, 2-12, 2-10, 2-8, 2-6, 2-3, 4-24, 4-20, 4-18, 4-12, 4-10, 4-8, 4-6, 6-24, 6-20, 6-18, 6-12, 6-10, 6-8, 8-24, 8-20, 8-18, 8-16, 8-12, 8-10, 10-24, 10-18, 10-16, 10-12, 12-24, 12-18, 12-16, 16-24, 16-20, 16-18, 18-24, 18-20, or 20-24 hours. The applying can crosslink the one or more ECM materials to the polymer scaffold. The final concentration of ECM material in the solution that is applied may be about 0.5, 0.6, 0.7, 0.8, 0.9, 1, 1.1, 1.2, 1.3, 1.4, or 1.5 w/v %. A range of final concentration of ECM material in the solution may be about 0.5-1.5, 0.5-1.2, 0.5-1, 0.5-0.07, 0.07-1.5, 0.07-1.2, 0.07-1, 1-1.5, 1-1.2, or 1.2-1.5 w/v %. The final concentration of crosslinker in the solution to be applied may be about 0.05, 0.06, 0.07, 0.08, 0.09, 0.1, 0.11, 0.12, 0.13, 0.15, 0.16, 0.17, 0.18, 0.19, or 0.2 v/v %. A range of final concentration of crosslinker in the solution may be about 0.05-0.2, 0.05-0.15, 0.05-0.1, 0.07-0.2, 0.07-0.1, or 0.1-0.2 v/v %.

Once the crosslinking has been completed, the excess coating solution may be washed (e.g., in any kind of water, including tertiary distilled water, distilled water without salt, and ultra-purified water without salt) from the scaffold substrate. The washing may occur 1, 2, 3, 4, 5, 6, 7, 8, 9, 10, or more times, including 1-10, 1-8, 1-6, 1-4, 1-2, 2-10, 2-8, 2-6, 2-4, 4-10, 4-8, 4-6, 6-10, 6-8, or 8-10 times, to remove the residual crosslinker contained in the scaffold. Residual crosslinker may cause toxicity in the human body, and there may be no more than 2 ppm of residential crosslinker in the scaffold. The washing can be performed for about 1, 2, 3, 4, 5, 6, 7, 8, 9, 10, 11, 12, 13, 14, 15, 16, 17, 18, 19, 20, 21, 22, 23, or 24 hours. Washing may be performed for may be performed for 1-24, 1-18, 1-12, 1-10, 1-8, 1-6, 1-2, 2-24, 2-18, 2-16, 2-12, 2-10, 2-8, 2-6, 2-3, 4-24, 4-20, 4-18, 4-12, 4-10, 4-8, 4-6, 6-24, 6-20, 6-18, 6-12, 6-10, 6-8, 8-24, 8-20, 8-18, 8-16, 8-12, 8-10, 10-24, 10-18, 10-16, 10-12, 12-24, 12-18, 12-16, 16-24, 16-20, 16-18, 18-24, 18-20, or 20-24 hours. Following washing, the scaffold is subject to freeze drying. This may be performed for about 1, 2, 3, 4, 5, 6, 7, 8, 9, 10, 11, 12, 13, 14, 15, 16, 17, 18, 19, 20, 21, 22, 23, or 24 or more hours. The freeze drying may be performed for 1-24, 1-18, 1-12, 1-10, 1-8, 1-6, 1-2, 2-24, 2-18, 2-16, 2-12, 2-10, 2-8, 2-6, 2-3, 4-24, 4-20, 4-18, 4-12, 4-10, 4-8, 4-6, 6-24, 6-20, 6-18, 6-12, 6-10, 6-8, 8-24, 8-20, 8-18, 8-16, 8-12, 8-10, 10-24, 10-18, 10-16, 10-12, 12-24, 12-18, 12-16, 16-24, 16-20, 16-18, 18-24, 18-20, or 20-24 hours. The manufacturing process may include two freeze-drying processes such that the first is performed for the crosslinking process and matrix formulation of natural polymers, and the second freeze-drying process may be performed for the re-drying of the scaffold after the residual crosslinker has been removed.

One or more therapeutic agents may be utilized in the scaffold such that the method of manufacturing may include applying one or more therapeutic agents directly or indirectly to part or all of the scaffold. The one or more therapeutic agents may be present in the polymer and 3D printed with the polymer as it is being 3D printed; it may be present in the crosslinking solution and/or with the one or more ECM materials; a combination of being printed with the polymer and being present in the crosslinking/ECM solution; and/or it may be applied to at least part of the outside of the scaffold following manufacture of the scaffold. The therapeutic agent(s) may be manufactured with the scaffold or otherwise placed thereon in such a manner that it may not stay on or with the scaffold but become part of the surrounding environment to facilitate the surrounding tissue with healing, cell infiltration to the scaffold, cell migration to the wound or diseased site, regeneration of cells or tissue, and so forth. The manufacturing process takes into consideration the concentration of therapeutic agent(s) for the scaffold when considering the amount to be therapeutically effective in surrounding soft tissue.

The manufacture of the scaffold may be designed such that degradation of the scaffold may be adjustable based on at least the concentration of the crosslinker, the concentration of the natural polymer decellularized extracellular matrix (dECM) solution, the type of crosslinker used, or a combination thereof. For patient requirements requiring or benefiting from a slower rate of degradation, a higher concentration of crosslinker and/or longer duration of crosslinking may be utilized during manufacture. For patient requirements that do not require a slower rate of degradation, a lower concentration of crosslinker may be utilized. The crosslinker concentration for standard manufacturing practices may be about 0.1%.

The scaffold may be printed into a defined, desired 2D or 3D shape, or each type may be used at a particular soft tissue site in need thereof. Examples of printed 2D shapes include, e.g., a line, a curve, a circle, a square, a crescent, a triangle, a rectangle, an oval, a trapezoid. Examples of 3D shapes include sphere, pyramid, cube, rectangular prism, cylinder, cone, triangular prism, bowl, or it may be customized to fit the wound or void (or both) of at the soft tissue site. In some cases for 2D shapes, a sheet may comprise one or more markings for one of more of the shapes.

Following manufacture, the produced scaffold may be sterilized and packaged to protect them from contamination by external microbes or the intrusion of organic bodies such as insects. The packaging may also eliminate the risk from pressure or shock within certain environments before use.

### V. Kits

Kits containing scaffolds of the disclosure or compositions to produce scaffolds of the disclosure, such as PCL, PDO, both PCL and PDO, one or more crosslinkers, and so forth, are encompassed in the disclosure.

Kits may comprise one or more components, any of which may be individually packaged or placed in a container, such as a package, tube, bottle, vial, syringe, or other suitable container means. The kit may comprise the scaffold or compositions to produce the scaffold, such as sealed in a package, including in a sterile environment, and the kit may optionally also include one or more therapeutic agents that are comprised in a tube, bottle, vial, syringe, etc. In some cases, the one or more therapeutic agents in the kit are comprised in one or more compositions to produce the scaffold.

Individual therapeutic agent components may be provided in a kit in concentrated amounts; a component may be provided individually in the same concentration as it would be in a solution with other components. Concentrations of components may be provided as 1×, 2×, 5×, 10×, or 20× or more, for example. Examples include at least hormone, antibiotic, pain reliever, hemorrheologic, vasoconstrictive, anti-inflammatory, anti-fibrotic, wound-healing agent, radioprotective material, anti-fungal, contraceptive, or any combination thereof.

The kit may be configured to allow for placement of the therapeutic agent on the scaffold at the point of care, or ahead of time of the point of care.

### VI. Examples

The following examples are included to demonstrate particular compositions and methods of the disclosure. It should be appreciated by those of skill in the art that the techniques disclosed in the examples that follow represent techniques discovered to function well in the practice of the methods and compositions of the disclosure, and thus can be considered to constitute particular modes for its practice. However, those of skill in the art should, in light of the present disclosure, appreciate that many changes can be made in the specific aspects that are disclosed and still obtain a like or similar result without departing from the spirit and scope of the subject matter of the disclosure.

### Example 1

### Soft Tissue Reinforcement Scaffold Characterization

The soft tissue reinforcement scaffold (which may be referred to as STRS), may be characterized by the following studies.

The scaffold may maintain one or more desired mechanical properties better than a control for at least a specific period of time, such as at least about 4, 6, 8, 10, 12, 24, 36, or 48 hours and including at least about 3, 4, 5, 6, or 7 days and including at least about 1, 2, 3, or 4 weeks and including at least about 1, 2, 3, 4, 5, 6, 7, 8, 9, 10, 11, or 12 months or more.

In one example of a mechanical property, one can characterize hydrolytic degradation. The scaffold was subject to 6 weeks of hydrolytic degradation in PBS at 37° C. In this test, mechanical properties were observed, such as Ultimate Tensile Strength (UTS) and Young's Modulus (YM), and microscopic images for degradation were taken at the time points: 0 and 6th weeks. The control group utilized was polyglactin 910 mesh (longitudinal direction) and the test scaffold was Polycaprolactone (PCL) STRS.

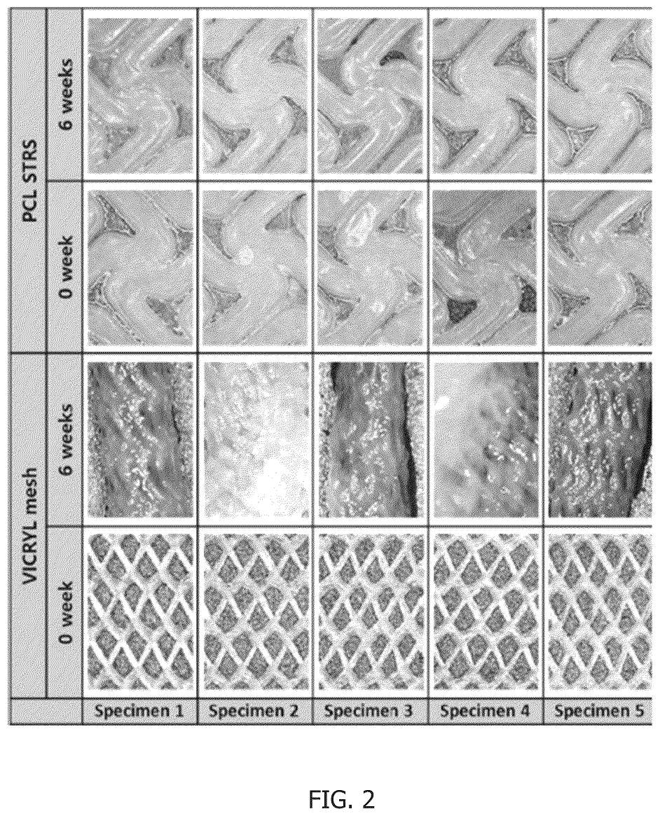

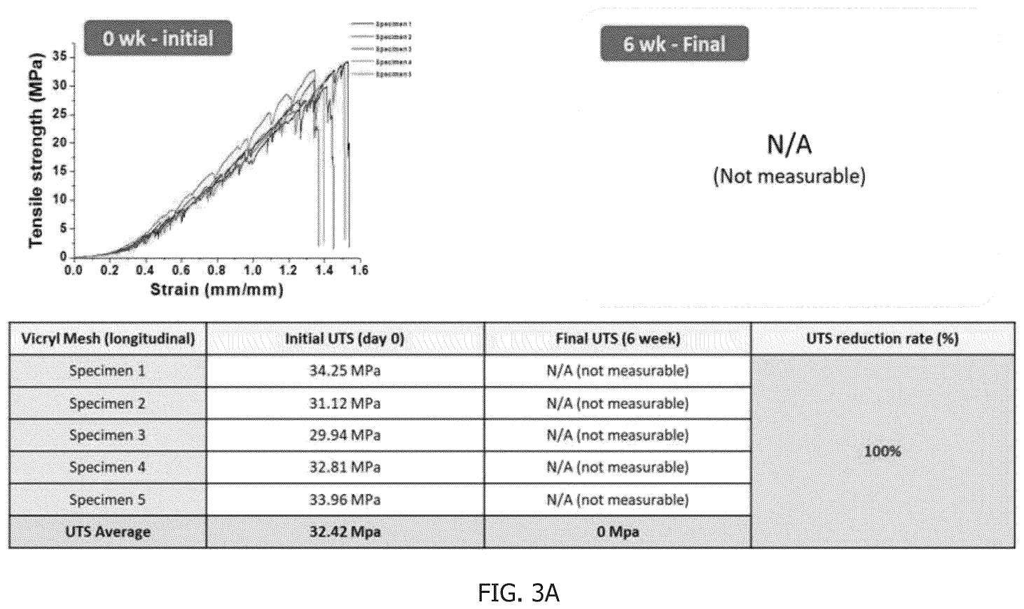

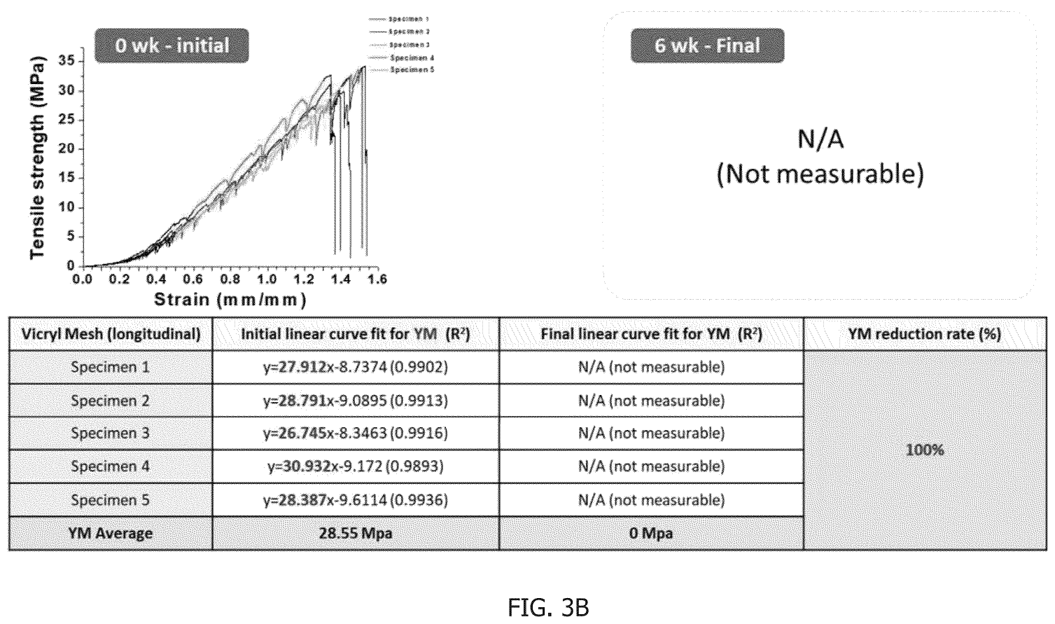

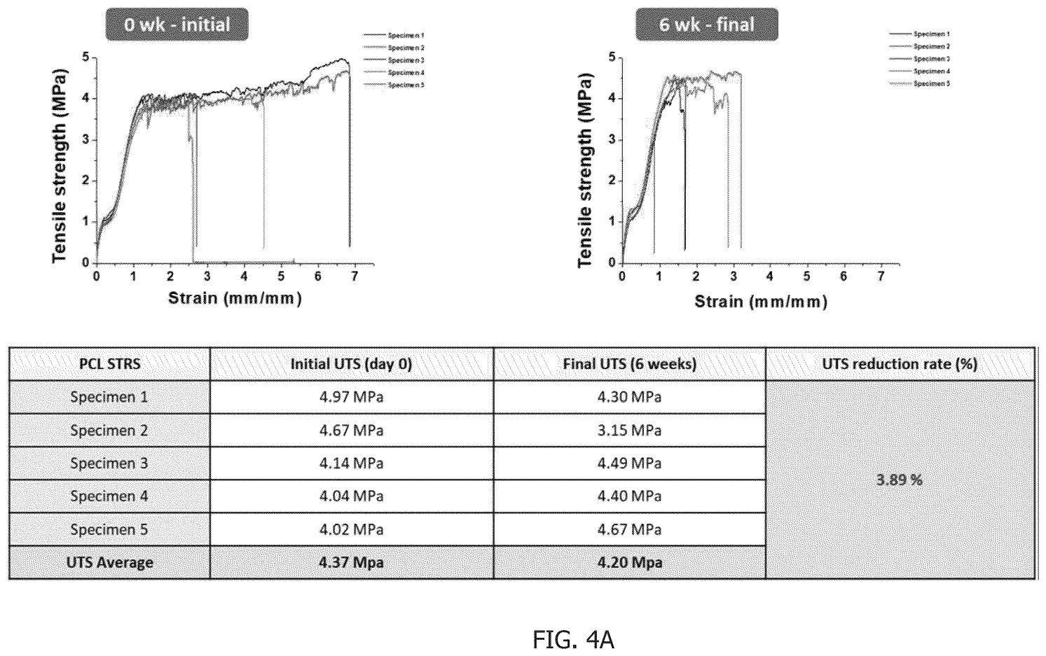

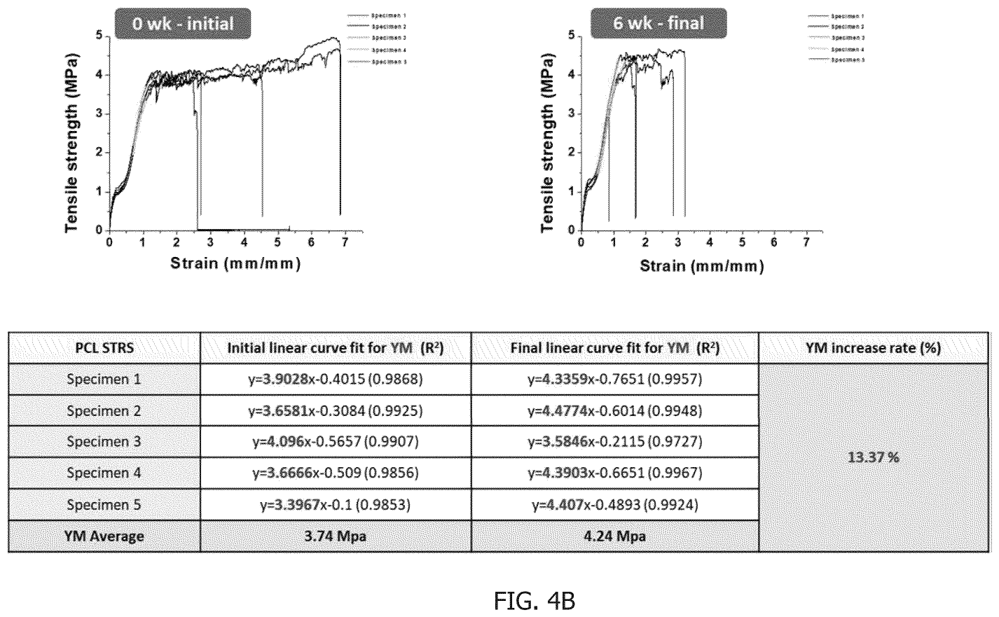

Microscopy results are provided in FIG. 2. The PCL STRS structure was kept in its original shape at the 6^{th} week. However, the structure of polyglactin 910 mesh was not maintained at the same timepoint. FIGS. 3A and 3B provide the degradation profile for the polyglactin 910 mesh, and FIGS. 4A and 4B provide the degradation profile for the PCL STRS structure. Both UTS and YM at the 6th week were regarded as 0 in the case of polyglactin 910 mesh, because they were not able to be measured (UTS, YM reduction rate (%)=100). However, PCL STRS maintained adequate UTS and YM for 6 weeks after degradation experiment. Thus, it is confirmed that PCL STRS can satisfy one example of a Critical to Quality (CTQ) criteria, and PCL is an appropriate material for manufacturing STRS.

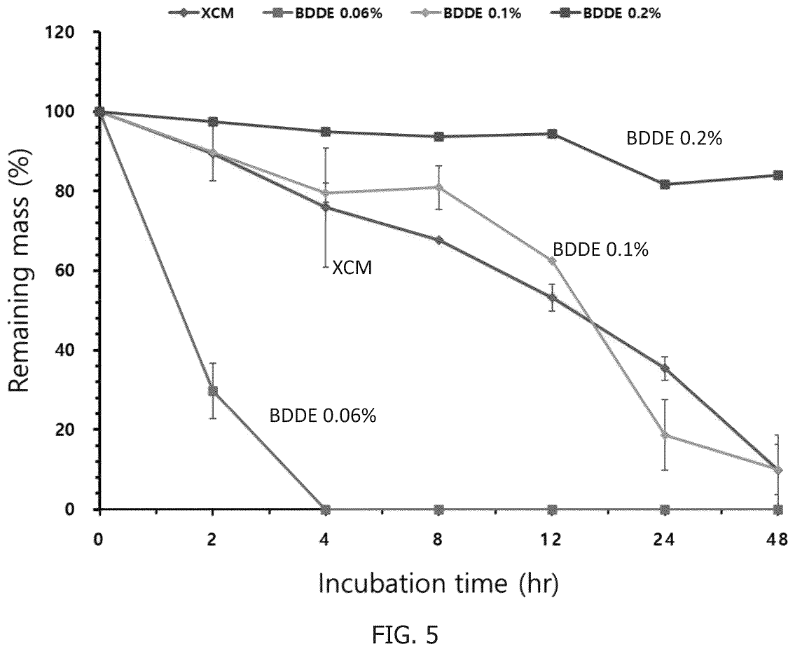

One example of a biological component of the STRS was characterized: enzymatic degradation. porcine-derived non-cross-linked extracellular collagen matrix was utilized as a control group for the enzymatic degradation test, and the degradation period was compared between porcine-derived non-cross-linked extracellular collagen matrix and collagen matrix made with difference concentrations of crosslinker 1,4-Butanediol Diglycidyl Ether (BDDE) using the same Units of collagenase. FIG. 5 shows the results of the degradation profile of porcine-derived non-cross-linked extracellular collagen matrix and natural polymer matrix produced using different concentrations of BDDE. The enzymatic degradation test was conducted using a collagenase of 100 U, and the value of porcine-derived non-cross-linked extracellular collagen matrix is the average of two different lots. The total sample size of XCM is nine, and sample size of BDDE concentration is five.

Comparing the degradation profile, matrix with a concentration of 0.06% BDDE had a faster degradation profile than porcine-derived non-cross-linked extracellular collagen matrix, and matrix with 0.1% concentration of BDDE had a degradation profile similar to porcine-derived non-cross-linked extracellular collagen matrix. Finally, matrix with 0.2% concentration of BDDE had a slower biodegradation period than porcine-derived non-cross-linked extracellular collagen matrix. In terms of biodegradation period, 0.1% concentration of BDDE and 0.2% concentration of BDDE may be utilized, as examples.

BDDE is a cytotoxic substance that can cause side effects in the human body, and the amount of residual BDDE may be controlled at less than 2 ppm (maximum acceptable per FDA requirements), and one may use as little as possible in consideration of safety. Furthermore, the cleaning process to meet the criteria may vary depending on the concentration of the initially used BDDE. Considering a variety of factors, a 0.1% concentration of BDDE with the same degradation profile as porcine-derived non-cross-linked extracellular collagen matrix was selected, and the number of washes for cleaning process of STRS was set as 5, as an example.

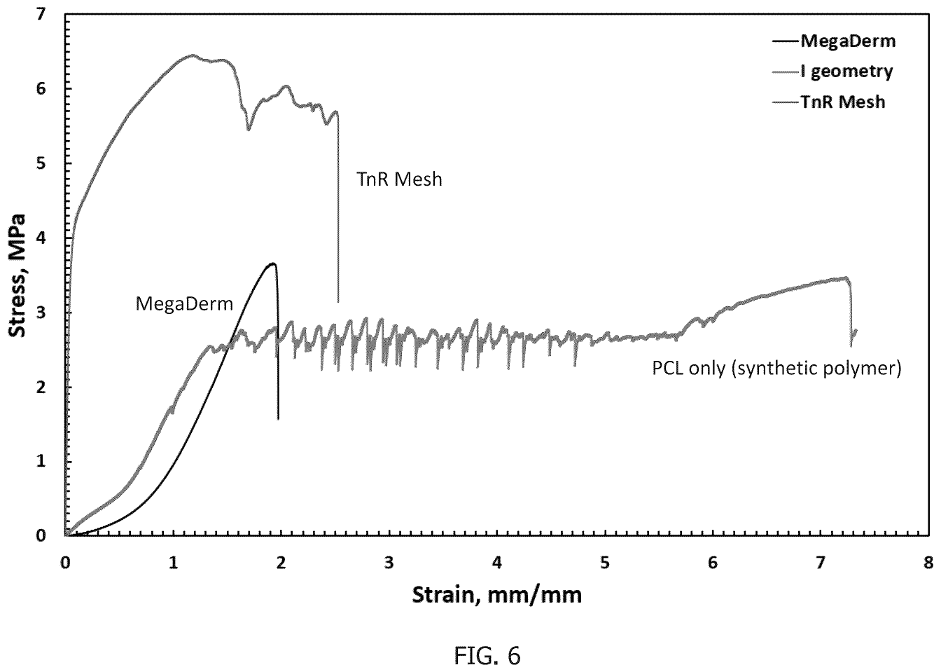

FIG. 6 provides a UTS graphic comparing the test STRS to commercial products MegaDerm (L&C BIO) (an Acellular Dermal Matrix (ADM) control) and TnR Mesh (T&R Biofab). The test STRS has the unit structure pattern encompassed by the claims (a unit structure generally resembling an “I”) is compared to a product having the same material but a different pattern of unit structure (TnR Mesh). Clearly, the test STRS has a curve more closely resembling the MegaDerm curve (ADM control) on the first 2 mm of the graphic. This curve resemblance, indicates that the STRS product behaves more closely to the ADM control, and therefore it is easier to replace it.

### Example 2

### Cell Infiltration

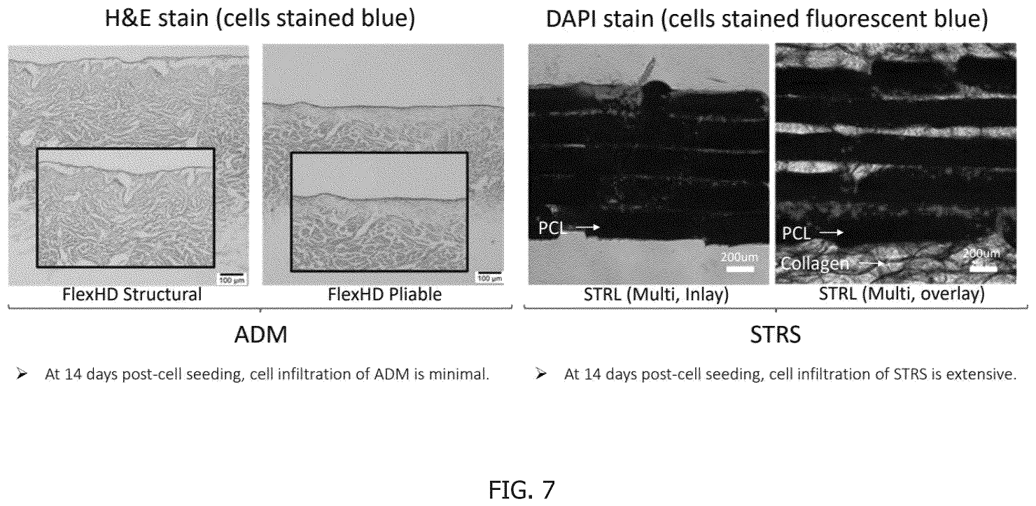

The STRS was compared to ADM products with respect to the ability to be infiltrated by cell growth. In the case of ADM products (representative ADM samples provided in H&E stain images of FIG. 7), cells could not penetrate the product because of the detailed internal structure of the product itself. However, in the case of the STRS product, it was manufactured to have a structure that allows cells to penetrate into the construct, and the image on the right of FIG. 7 for the in vitro test confirms this. This indicates that when transplanting a product into an individual, it is easy for cells to penetrate and self-organize.

### Example 3

### Control of Degradation

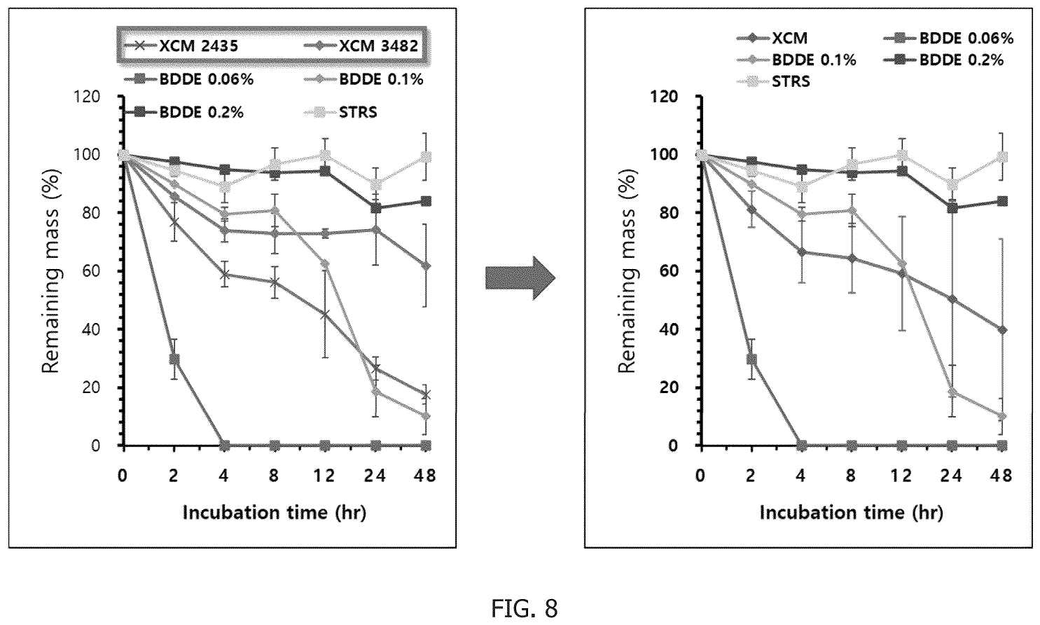

The STRS product may comprise natural polymer(s). In such a case, one can control the degradation period of the natural polymer(s) by adjusting the concentration of the crosslinker. The STRS product can have similar, higher, or lower degradation resistance when compared to ADM, which was confirmed through collagenase degradation test in vitro (FIG. 8). This suggests that the physical properties of the product can be adjusted according to the indication to use the product.

### Example 4

### Manufacture of STRS

An example of one demonstration of overall STRS manufacture process is provided. The manufacture process is designed to consider performance factors, such as tensile strength and strain; stability, such as being heat-stable and resistant to solvent; and printability (having a productive print head speed). synthetic polymers were examined according to each detailed factor. Testing of a variety of synthetic polymers resulted in selection of Polycaprolactone and Polydioxanone for further characterization. Various pattern design candidates were selected for STRS fabrication, some of which have similar values of UTS and SUTS, and a similar stress train curve as ADMs. As a result of tensile tests using these polymers, a final pattern having a much lower value of UTS than ADM was selected, which was the pattern having a unit pattern generally in the shape of “I.” Following UTS analysis of multiple unit patterns as a function of varying line width, and considering the texture of the scaffold, one may utilize a STRS having LW of about 500 μm with 18 ea/cm 2 pattern density for PCL and/or a STRS having LW 400 μm with 25 ea/cm 2 pattern density for PDO.

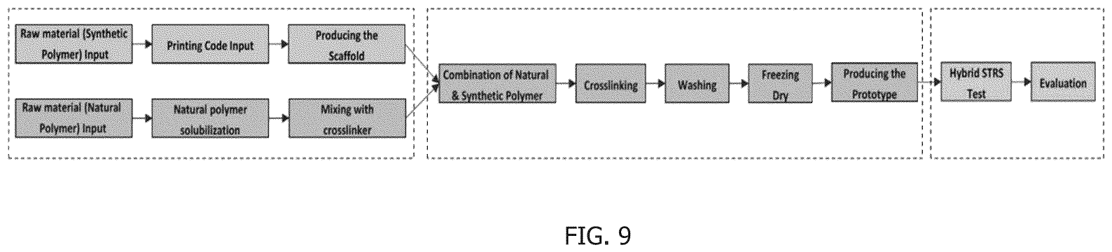

STRS was manufactured using an inlay method comprising a multi-layer scaffold (with a thickness of 0.2 mm scaffold×5 sheets) of the PCL (Pattern P) and PDO (Pattern P) candidates described above, respectively. This included a tendon decellularized extracellular matrix (dECM) solution as the natural polymer and BDDE as the crosslinker. One example of an overall STRS manufacturing process is provided in FIG. 9.

1. Prepare the Synthetic Polymer Scaffolds

Scaffolds are manufactured under the conditions described in the previous sections.

2. Prepare the Mixture of Natural Polymer and Crosslinker
a) Put the Tendon dECM in 0.01N HCl at 1.2 w/v % and swell it at 4° C. for 4-5 hours.
b) Grind the swelled tendon dECM with a homogenizer.

(The grinding condition is 6000 rpm and after grinding for one minute, leave it on ice for 30 seconds to prevent temperature rise. Repeat five times in total.)
c) Store the tendon dECM solution at 4° C. before use and use within 24 hours.
d) Dilute the tendon dECM solution made of 1.2% with 0.01N HCl containing BDDE. At this time, mix the final concentration to 1 w/v % tendon dECM solution and 0.1 v/v % BDDE.
e) To remove the bubbles in the mixture, centrifuge the mixture at 4° C. for 5 minutes at 2000 rpm.
f) To remove invisible particles, filter the mixture with a 500 um sieve.
g) To remove bubbles, perform the centrifugation process described in No. 5 repeatedly.

3. Combination of Natural & Synthetic Polymer

The process involves integrating prepared scaffolds and a mixture of natural polymers and BDDE, as described:
a) Insert the membrane paper into the lower frame
b) Load 5 ml of the prepared mix onto the membrane paper.
c) Position the scaffolds (5 individual sheets) on the loaded mixture.
d) Using a scraper, spread the mixture located below the multi-layer scaffold to fill all the pores of the scaffold.
e) Place a membrane paper on top of the multi-layer scaffold.
f) Combine the upper frame to remove the remaining mixture. In order to guarantee the uniform thickness of the STRS, the upper frame should be strongly joined to lower frame.

4. Crosslinking (Freeze-Drying Process)

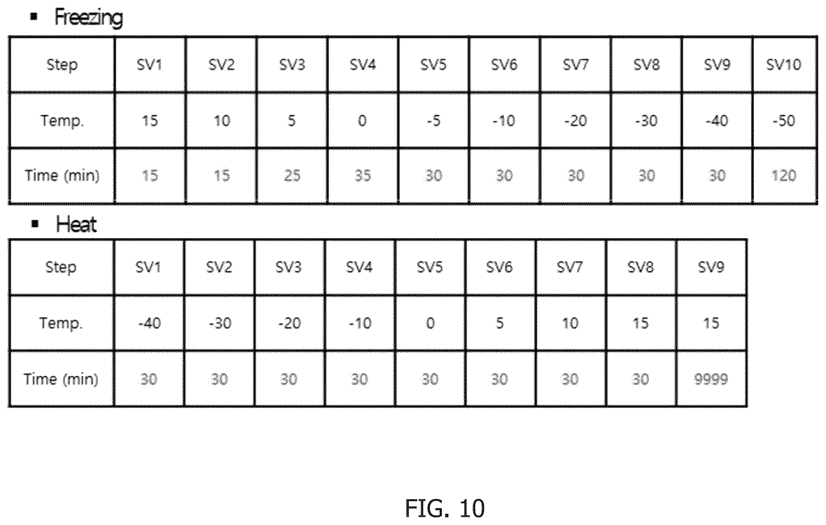

The natural polymer is integrated with the synthetic polymer as it is crosslinked by the BDDE. The natural polymer is crosslinked through the freeze-drying process.
a) Put the sample prepared in the combination of natural & synthetic polymer into the freeze dryer.
b) Freeze drying is carried out under suitable conditions. One example is provided in FIG. 10.

5. Washing

This process is to remove residual crosslinker after the crosslinking process of natural polymer.
a) After the freeze drying is completed, separate the STRS from the frame.
b) Place the tray on the orbital shaker as shown on the right side of the picture above and put STRS in it.
c) Add 100 ml of distilled water (D.W.) per STRS. (STRS was based on 75×75 mm. If the size increases, the amount of D.W. needs to be adjusted accordingly.)
d) Rotate at room temperature for 10 minutes at 30-50 rpm.
e) Replace with fresh D.W. and perform the rotating and replacing with fresh D.W. 5 times in total.

6. Freeze-Drying
a) Put the washed STRS into the frame or plasticware and put it in the freeze dryer.
b) Freeze-drying is carried out under suitable conditions (e.g., see FIG. 10).

The manufactured STRS product may be packed and stored, or used substantially immediately.

### Example 5

### Tensile Tests of Manufactured STRS of PCL Vs. PDO

A STRS of PCL at a line width of 400 μm and a STRS of PDO having a line width of 500 μm, each having a unit structure generally in the shape of “I,” were characterized for tensile strength.

The tensile test was conducted with three types of STRS prototype made of scaffold with PCL 500 μm, and PDO 400 μm line width, the results of which can be seen in Table 1. As can be seen in the table, 4.40 MPa was measured for PCL 500 μm, and each STRS had a lower value than the previous synthetic polymer scaffolds. However, as can be seen in Table 2, which is the result of the ADM tensile test, PCL 500 μm is higher than the MegaDerm and little lower than the XCM.

TABLE 1
Tensile Test Result and Information of STRS

| Pattern | P | | | |
|---|---|---|---|---|
| Shape | [symbol] | | | |
| Line Width | 500 | μm | 400 | μm |
| Thickness | 0.9-1 mm | | | |
| Material | Evonik PCL | | Evonik PDO | |
| Sample Type | Polymer only | STRS | Polymer only | STRS |
| Stress, MPa | 4.71 | 4.40 | 6.13 | 3.34 |
| Strain, mm/mm | 5.81 | 1.48 | 2.81 | 1.36 |
| Sample Size | 3 | | 3 | |
| Pattern Density | 18 | ea/cm² | 25 | ea/cm² |

| | |
|---|---|
| Test Condition | 1. Strain rate: 5 mm/min |
| | 2. Specimen size: 20 × 50 cm |
| | 3. Hydrated in 37° C. PBS 1 X for 1 min(Time for the collagen of STRS is fully hydrated) |

TABLE 2
Tensile Test Result and Information of ADMs

| | FlexHD | FlexHD | | | | | |
|---|---|---|---|---|---|---|---|
| | Pliable | Pliable | FlexHD | | | XCM | |
| ADM | SF 2002 | AP 1616 | Structural | AlloDerm | AlloMend | BIOLOGIC | MegaDerm |
| Stress, | 12.6 | 3.40 | 10.53 | 6.48 | 5.67 | 5.18 | 3.76 |
| MPa | | | | | | | |
| Strain, | 3.41 | 3.38 | 2.69 | 4.00 | 2.56 | 1.37 | 2.11 |
| mm/mm | | | | | | | |
| Sample | 3 | 7 | 5 | 5 | 5 | 5 | 15 |
| size | | | | | | | |

| | |
|---|---|
| Test | 1. Strain rate: 10 mm/min (Follows ASTM D 638-14) |
| Condition | 2. Specimen size: 3.18 × 7.62 mm (Follows ASTM D 638-14) |
| | 3. Hydrated in 37° C. PBS 1 × for 40 mins(Time for the temperature of the ADM reaches 37° C.) |

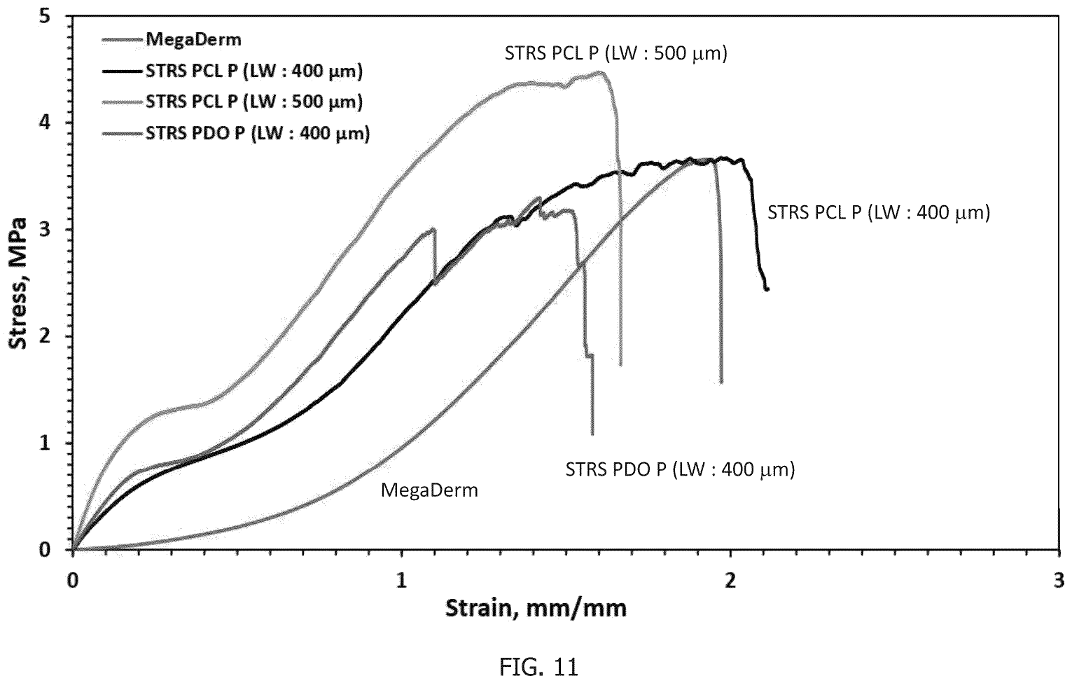

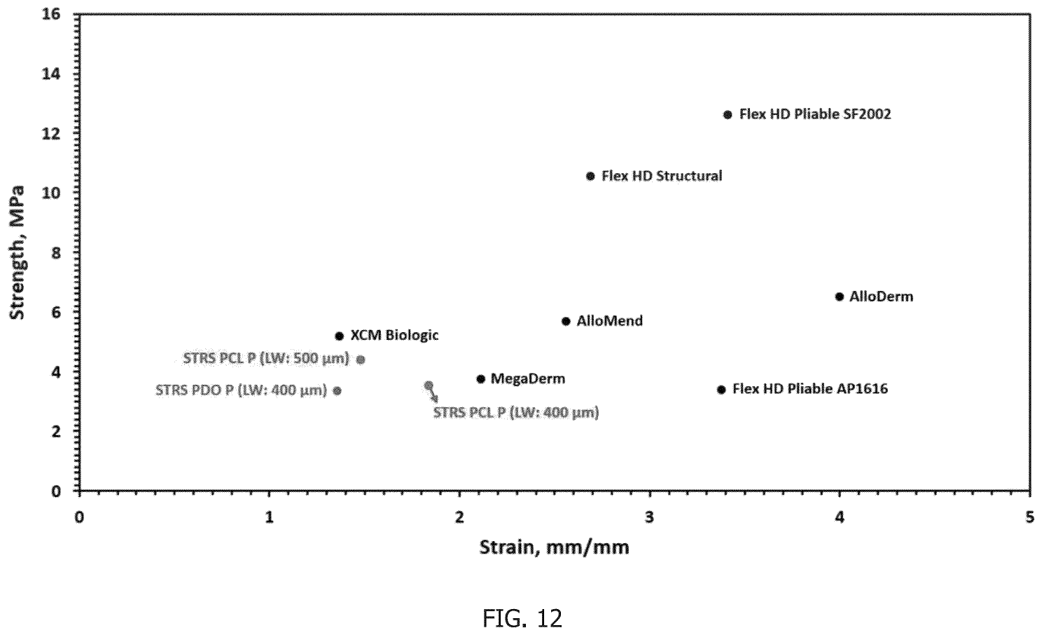

FIG. 11 provides a UTS image for STRS of different types and line widths compared to MegaDerm. The PCL STRS having line width of 400 um more closely approximated the ADM control, MegaDerm. FIG. 12 provides a Bubble Chart of STRS and MegaDerm, also reflecting that PCL STRS having line width of 400 um more closely approximated the ADM control. One may further characterize the PCL STRS or the PDO STRS using burst test, tear test, cell infiltration assay, etc.

### VII. Recitation of Embodiments

Embodiment 1. An artificial support structure comprising one or more biodegradable polymers and one or more extracellular matrix materials, the support structure further comprising: a plurality of unit patterns, each unit pattern comprising a plurality of filaments, arranged continuously, symmetrically, and regularly thereon, each unit pattern being constituted of edges of a closed shape to thus form a pore at the inside thereof, wherein the plurality of unit patterns is connected to thus have intersection points with one another and the number of intersection points is the same as the number of edges passing the intersection points.
Embodiment 2. The artificial support structure of Embodiment 1, wherein the one or more extracellular matrix materials comprises Collagen I.
Embodiment 3. The artificial support structure of Embodiment 1 or 2, wherein at least one unit pattern has a diameter of between about 200 microns to about 3.5 mm.
Embodiment 4. The artificial support structure of any of Embodiments 1-3, wherein at least one unit pattern has a diameter of between about 1.5 mm to about 3 mm.
Embodiment 5. The artificial support structure of any of Embodiments 1-4, wherein at least one unit pattern has a diameter of between about 1.782 mm to about 2.97 mm.
Embodiment 6. The artificial support structure of any of Embodiments 1-5, wherein the biodegradable polymer material further comprises one or more extracellular matrix material.
Embodiment 7. The artificial support structure of any of Embodiments 1-6, wherein the one or more extracellular matrix materials is Collagen I.
Embodiment 8. The artificial support structure of any of Embodiments 1-7, wherein the plurality of connected unit patterns form a substantially planar sheet.
Embodiment 9. The artificial support structure of any of Embodiments 1-8, wherein the plurality of connected unit patterns form a three-dimensional macrostructure.
Embodiment 10. The artificial support structure of any of Embodiments 1-9, wherein the artificial support structure has a thickness between about 0.5 mm to about 1.5 mm.
Embodiment 11. The artificial support structure of any of Embodiments 1-10, wherein the artificial support structure has a thickness between about 0.7 mm to about 1.3 mm.
Embodiment 12. The artificial support structure of any of Embodiments 1-11, wherein the artificial support structure has a thickness between about 0.9 mm to about 1.1 mm.
Embodiment 13. The artificial support structure of any of Embodiments 1-12, wherein the artificial support structure contains between 1 and 5 layers.
Embodiment 14. The artificial support structure of Embodiment 13, wherein the layers have further thicknesses of between 0.10 mm to about 0.3 mm.
Embodiment 15. The artificial support structure of Embodiment 13 or 14, wherein the layers have further thicknesses of between 0.15 mm to about 0.25 mm.
Embodiment 16. The artificial support structure of any of Embodiments 13-15, wherein the layers have further thicknesses of between 0.18 mm to about 0.22 mm.
Embodiment 17. The artificial support structure of any of Embodiments 1-16, wherein one or more filaments in the plurality of filaments have a diameter of less than 550 microns.
Embodiment 18. The artificial support structure of any of Embodiments 1-17, wherein one or more filaments in the plurality of filaments have a diameter of less than 500 microns.
Embodiment 19. The artificial support structure of any of Embodiments 1-18, wherein one or more filaments in the plurality of filaments have a diameter of less than 400 microns.
Embodiment 20. The artificial support according to any of Embodiments 1-19, wherein four-unit patterns are connected to have four intersection points with one another and have four edges passing the four intersection points, and a space surrounded with the four-unit patterns has the same or similar shape as or to each unit pattern.
Embodiment 21. The artificial support according to any of Embodiments 1-20, wherein each unit pattern is an uppercase “I” of the English alphabet.
Embodiment 22. The artificial support according to Embodiment 21, wherein short edges of the edges of the closed shape of each unit pattern have the same length as one another, and long edges thereof have the same length as one another, so that the space surrounded with the four-unit patterns has the same shape as each unit pattern.
Embodiment 23. The artificial support according to Embodiment 22, wherein, in the closed shape, a length ratio of the short edge to the long edge is 1:3.
Embodiment 24. The artificial support structure of any of Embodiments 1-23, wherein the artificial support structure has an ultimate tensile strength of between about 4 MPa to about 5 MPa.
Embodiment 25. The artificial support structure of any of Embodiments 1-24, wherein the artificial support structure has an ultimate tensile strength of between about 4.05 MPa to about 4.7 MPa.
Embodiment 26. The artificial support structure of any of Embodiments 1-25, wherein the artificial support structure has an ultimate tensile strength of between about 4.12 MPa to about 4.50 MPa.
Embodiment 27. The artificial support structure of any of Embodiments 1-26, wherein the artificial support structure has a modulus of elasticity of about 2.8 MPa to about 4.2 MPa.
Embodiment 28. The artificial support structure of any of Embodiments 1-27, wherein the artificial support structure has a modulus of elasticity of about 3.00 MPa to about 4.10 MPa.
Embodiment 29. The artificial support structure of any of Embodiments 1-28, wherein the artificial support structure has a modulus of elasticity of about 3.06 Mpa to about 4.00 Mpa.
Embodiment 30. The artificial support structure of any of Embodiments 1-29, wherein the artificial support structure has a suture retention strength of about 20 N to about 26 N.
Embodiment 31. The artificial support structure of any of Embodiments 1-30, wherein the artificial support structure has a suture retention strength of about 21 N to about 25 N.
Embodiment 32. The artificial support structure of any of Embodiments 1-31, wherein the artificial support structure has a suture retention strength of about 22.03 N to about 24.27 N.
Embodiment 33. The artificial support structure of any of Embodiments 1-32, wherein the artificial support structure has a burst strength of about 140 N to about 170 N.
Embodiment 34. The artificial support structure of any of Embodiments 1-33, wherein the artificial support structure has a burst strength of about 145 N to about 163 N.
Embodiment 35. The artificial support structure of any of Embodiments 1-34, wherein the artificial support structure has a burst strength of about 147.14 N to about 161.26 N.
Embodiment 36. The artificial support structure of any of Embodiments 1-35, wherein the artificial support structure has a tear resistance of about 18 N to about 26 N.
Embodiment 37. The artificial support structure of any of Embodiments 1-36, wherein the artificial support structure has a tear resistance of about 19 N to about 25 N.
Embodiment 38. The artificial support structure of any of Embodiments 1-37, wherein the artificial support structure has a tear resistance of about 19.87 N to about 24.92 N.
Embodiment 39. An artificial support structure comprising one or more biodegradable polymers and one or more extracellular matrix materials, the support structure further comprising: a plurality of unit patterns, each unit pattern comprising a plurality of filaments, arranged repeatedly to constitute columns or rows symmetrical to one another, each unit pattern being constituted of edges of a closed shape to thus form a pore at the inside thereof, wherein the columns or rows along which the plurality of unit patterns are arranged repeatedly have an Eulerian trail.
Embodiment 40. The artificial support structure of Embodiment 39, wherein the one or more extracellular matrix materials comprises Collagen I.
Embodiment 41. The artificial support structure of Embodiments 39 or 40, wherein at least one unit pattern has a diameter of between about 200 microns to about 3.5 mm.
Embodiment 42. The artificial support structure of Embodiment 41, wherein at least one unit pattern has a diameter of between about 1.5 mm to about 3 mm.
Embodiment 43. The artificial support structure of Embodiment 42, wherein at least one unit pattern has a diameter of between about 1.782 mm to about 2.97 mm
Embodiment 44. The artificial support structure of any of Embodiments 39-43, wherein the biodegradable polymer material further comprises one or more extracellular matrix materials.
Embodiment 45. The artificial support structure of any of Embodiments 39-44, wherein the one or more extracellular matrix materials is Collagen I.
Embodiment 46. The artificial support structure of any of Embodiments 39-45, wherein the plurality of connected unit patterns form a substantially planar sheet.
Embodiment 47. The artificial support structure of any of Embodiments 39-46, wherein the plurality of connected unit patterns form a three-dimensional macrostructure.
Embodiment 48. The artificial support structure of Embodiments 39-47, wherein the artificial support structure has a thickness between about 0.5 mm to about 1.5 mm.
Embodiment 49. The artificial support structure of any of Embodiments 39-48, wherein the artificial support structure has a thickness between about 0.7 mm to about 1.3 mm.
Embodiment 50. The artificial support structure of any of Embodiments 39-40, wherein the artificial support structure has a thickness between about 0.9 mm to about 1.1 mm.
Embodiment 51. The artificial support structure of any of Embodiments 39-50, wherein the artificial support structure contains between 1 and 5 layers.
Embodiment 52. The artificial support structure of Embodiment 51, wherein the layers have further thicknesses of between 0.10 mm to about 0.3 mm.
Embodiment 53. The artificial support structure of any of Embodiments 51 or 52, wherein the layers have further thicknesses of between 0.15 mm to about 0.25 mm.
Embodiment 54. The artificial support structure of any of Embodiments 51-53, wherein the layers have further thicknesses of between 0.18 mm to about 0.22 mm.
Embodiment 55. The artificial support structure of any of Embodiments 39-54, wherein one or more filaments in the plurality of filaments has a diameter of less than 550 mm.
Embodiment 56. The artificial support structure of any of Embodiments 39-55, wherein one or more filaments in the plurality of filaments has a diameter of less than 500 mm.
Embodiment 57. The artificial support structure of any of Embodiments 39-56, wherein one or more filaments in the plurality of filaments has a diameter of less than 400 mm.
Embodiment 58. The artificial support according to any of Embodiments 39-57, wherein columns or rows along which the plurality of unit patterns are arranged repeatedly are connected to allow the plurality of unit patterns to have intersection points with the plurality of unit patterns of the neighboring columns or rows.
Embodiment 59. The artificial support according to Embodiment 58, wherein the plurality of unit patterns have the number of intersection points that is the same as the number of edges passing the intersection points.
Embodiment 60. The artificial support according to Embodiment 59, wherein four neighboring unit patterns are connected to have four intersection points with one another and have four edges passing the four intersection points, and a space surrounded with the four-unit patterns has the same or similar shape as or to each unit pattern.
Embodiment 61. The artificial support according to any of Embodiments 39-60, wherein each unit pattern is an uppercase “I” of the English alphabet.
Embodiment 62. The artificial support according to Embodiment 61, wherein short edges of the edges of the closed shape of each unit pattern have the same length as one another, and long edges thereof have the same length as one another, so that the space surrounded with the four-unit patterns has the same shape as each unit pattern.
Embodiment 63. The artificial support according to Embodiments\ 62, wherein, in the closed shape, length ratio of a short edge to a long edge is 1:3.
Embodiment 64. The artificial support according to Embodiment 63, wherein, in the closed shape, a length ratio of each short edge to each long edge is 1:3.
Embodiment 65. The artificial support according to Embodiment 64, wherein the plurality of unit patterns in the columns or rows along which the plurality of unit patterns is repeatedly arranged have an angle of 45° or 135° with respect to the rows or columns.
Embodiment 66. The artificial support according to Embodiment 65, wherein portions of the edges of the plurality of unit patterns are arranged regularly to form the edges of the artificial support.
Embodiment 67. The artificial support structure of any of Embodiments 39-66, wherein the artificial support has an ultimate tensile strength of between about 4.05 MPa to about 4.7 MPa.
Embodiment 68. The artificial support structure of any of Embodiments 39-67, wherein the artificial support has an ultimate tensile strength of between about 4.12 MPa to about 4.50 MPa.
Embodiment 69. The artificial support structure of any of Embodiments 39-68, wherein the artificial support structure has a modulus of elasticity of about 2.8 MPa to about 4.2 MPa.
Embodiment 70. The artificial support structure of any of Embodiments 39-69, wherein the artificial support structure has a modulus of elasticity of about 3.00 MPa to about 4.10 MPa.
Embodiment 71. The artificial support structure of any of Embodiments 39-70, wherein the artificial support structure has a modulus of elasticity of about 3.06 MPa to about 4.00 MPa.
Embodiment 72. The artificial support structure of any of Embodiments 39-71, wherein the artificial support structure has a suture retention strength of about 20 N to about 26 N.
Embodiment 73. The artificial support structure of any of Embodiments 39-72, wherein the artificial support structure has a suture retention strength of about 21 N to about 25 N.
Embodiment 74. The artificial support structure of any of Embodiments 39-73, wherein the artificial support structure has a suture retention strength of about 22.03 N to about 24.27 N.
Embodiment 75. The artificial support structure of any of Embodiments 39-74, wherein the artificial support structure has a burst strength of about 140 N to about 170 N.
Embodiment 76. The artificial support structure of any of Embodiments 39-75, wherein the artificial support structure has a burst strength of about 145 N to about 163 N.
Embodiment 77. The artificial support structure of any of Embodiments 39-76, wherein the artificial support structure has a burst strength of about 147.14 N to about 161.26 N.
Embodiment 78. The artificial support structure of any of Embodiments 39-77, wherein the artificial support structure has a tear resistance of about 18 N to about 26 N.
Embodiment 79. The artificial support structure of any of Embodiments 39-78, wherein the artificial support structure has a tear resistance of about 19 N to about 25 N.
Embodiment 80. The artificial support structure of any of Embodiments 39-79, wherein the artificial support structure has a tear resistance of about 19.87 N to about 24.92 N.
Embodiment 81. A scaffold, comprising a patterned polymeric substrate having a coating of one or more extracellular matrix (ECM) materials thereon, wherein the pattern of the polymeric substrate comprises adjacent rows of a series of unit cell structures each generally in the shape of the letter “I”, the unit cell structures aligned in the rows of the patterned polymeric substrate in a perpendicularly alternating pattern of the unit cell structures.
Embodiment 82. The scaffold of Embodiment 81 wherein the unit cell structures of the series are further defined as comprising a pore shaped as a central line greater in length than two substantially equal-length lines each perpendicular to opposite ends of the central line, and wherein the alternating pattern is configured such that each of the ends of the central line of the pore are generally perpendicular to a central line of a pore of an adjacent unit cell structure.
Embodiment 83. The scaffold of Embodiments 81 or 82, wherein the scaffold is configured as one or more sheets, each comprising a first planar side and a second planar side.
Embodiment 84. The scaffold of Embodiment 83, wherein the scaffold comprises 1, 2, 3, 4, or 5 sheets, or comprises at least or no more than 1, 2, 3, 4, or 5 sheets.
Embodiment 85. The scaffold of Embodiments 83 or 84, wherein the multiple sheets are configured such that a planar side of one sheet is adjacent to a planar side of another sheet.
Embodiment 86. The scaffold of any of Embodiments 82-85, wherein the scaffold comprises one or more a defined shape.
Embodiment 87. The scaffold of Embodiment 86, wherein the defined shape is generally a line, a curve, a circle, a square, a crescent, a triangle, a rectangle, an oval, a trapezoid, or wherein the scaffold comprises markings for one of more of said defined shapes.
Embodiment 88. The scaffold of any of Embodiments 81-87, wherein the polymeric substrate comprises Polycaprolactone, Polydioxanone, or a combination thereof.
Embodiment 89. The scaffold of any of Embodiments 81-88, wherein the polymeric substrate is comprised of Polycaprolactone.
Embodiment 90. The scaffold of any of Embodiments 81-89, wherein the one or more ECM materials comprise a single type of collagen or a combination of one or more types of collagen.
Embodiment 91. The scaffold of Embodiment 90, wherein the collagen is sourced from tendon, rat tail, bovine, porcine, or is recombinant.
Embodiment 92. The scaffold of Embodiment 90 or 91, wherein the combination of one or more types of collagen comprises Type I collagen and Type III collagen.
Embodiment 93. The scaffold of Embodiment 92, wherein the collagen is telocollagen derived from bovine tendon, rat tail tendon, or is recombinant.
Embodiment 94. The scaffold of any of Embodiments 81-93, wherein the coating is comprised on the first side of the sheet, the second side of the sheet, or both the first and second sides of the sheet.
Embodiment 95. The scaffold of any of Embodiments 81-94, wherein the coating fills the pore of multiple unit cell structures of the scaffold.
Embodiment 96. The scaffold of any of Embodiments 81-95, wherein the coating fills the pore of the majority of unit cell structures of the scaffold.
Embodiment 97. The scaffold of any of Embodiments 81-96, wherein the coating fills the pore of substantially all unit cell structures of the scaffold.
Embodiment 98. The scaffold of any of Embodiments 81-97, wherein the coating does not fill the pore of the majority of unit cell structures of the scaffold.
Embodiment 99. The scaffold of any of Embodiments 81-98, wherein the coating does not fill the pore of substantially all unit cell structures of the scaffold.
Embodiment 100. The scaffold of any of Embodiments 81-99, wherein the thickness of the scaffold is not greater than 1 mm.
Embodiment 101. The scaffold of any of Embodiments 81-100, wherein the scaffold further comprises one or more therapeutic agents.
Embodiment 102. The scaffold of Embodiment 101, wherein the one or more therapeutic agents comprises one or more growth factors, one or more cytokines, one or more chemokines, one or more drugs, or a combination thereof.
Embodiment 103. A method of producing the scaffold of any of Embodiments 81-102, the method comprising: (a) three-dimensionally printing the patterned polymeric substrate; (b) applying the one or more ECM materials to the substrate; (c) subjecting the substrate to one or more crosslinkers; (d) optionally washing the substrate; and (e) subjecting the substrate to conditions less than 15° C. in temperature, optionally wherein the one or more ECM materials and the one or more crosslinkers are mixed together prior to applying to the substrate.
Embodiment 104. The method of Embodiment 103, wherein the polymeric substrate is comprised of Polycaprolactone, Polydioxanone, or a combination thereof.
Embodiment 105. The method of Embodiment 103 or 104, wherein the applying comprises submerging the substrate in a solution of the coating.
Embodiment 106. The method of any of Embodiments 103-105, wherein the applying comprises laying the substrate on top of a solution of the coating.
Embodiment 107. The method of any Embodiments of 103-106, wherein the applying comprises spraying, dropping onto, or laying the coating onto the substrate.
Embodiment 108. The method of any of Embodiments 107-111, wherein the applying is performed for 1-24 hours, 5-24 hours, 5-20 hours, 8-20 hours, 8-15 hours, or 9-11 hours.
Embodiment 109. The method of any of Embodiments 103-108, wherein the one or more crosslinkers is selected from the group consisting of 1,4-Butanediol Diglycidyl Ether (BDDE), hexamethylene diisocyanate (HDMI), glutaraldehyde (GA), genipin, and a combination thereof
Embodiment 110. The method of any of Embodiments 103-109, wherein (c) is performed for about 1, 2, 3, 4, 5, 6, 7, 8, 9, 10, 11, 12, 13, 14, 15, 16, 17, 18, 19, 20, 21, 22, 23, or 24 hours.
Embodiment 111. The method of any of Embodiments 103-110, wherein the washing occurs with water.
Embodiment 112. The method of any of Embodiments 103-111, wherein (d) is performed for about 1, 2, 3, 4, 5, 6, 7, 8, 9, 10, 11, 12, 13, 14, 15, 16, 17, 18, 19, 20, 21, 22, 23, or 24 hours.
Embodiment 113. The method of any of Embodiments 103-112, wherein (e) is performed for about 1, 2, 3, 4, 5, 6, 7, 8, 9, 10, 11, 12, 13, 14, 15, 16, 17, 18, 19, 20, 21, 22, 23, or 24 hours.
Embodiment 114. The method of any of Embodiments 103-113, wherein following (e), the produced scaffold is subject to drying.
Embodiment 115. The method of any of Embodiments 103-114, further comprising applying one or more therapeutic agents to the scaffold.
Embodiment 116. The method of Embodiment 115, wherein the one or more therapeutic agents are present in the polymer, the coating, both the polymer and the coating, and/or are applied to at least part of the outside of the scaffold.
Embodiment 117. The method of any of Embodiments 103-116, wherein degradation of the patterned polymeric substrate is adjustable based on the concentration of the crosslinker.
Embodiment 118. The method of any of Embodiments 103-117, comprising washing the substrate after subjecting the substrate to one or more crosslinkers.
Embodiment 119. The method of any of Embodiments 103-117, wherein the scaffold is produced in a defined shape.
Embodiment 120. The method of Embodiment 119, wherein the defined shape is configured based on formation by a mandrel or is a pre-programmed macrostructure that is 3D printed.
Embodiment 121. A method of reinforcing soft tissue in an individual in need thereof, comprising applying an effective amount of the scaffold of any of Embodiments 1-102 to one or more soft tissue sites of the individual.
Embodiment 122. The method of Embodiment 121, wherein the soft tissue comprises muscle, tendon, ligament, fascia, fat, skin, nerve, blood vessel, or a combination thereof.
Embodiment 123. The method of Embodiment 121 or 122, wherein the soft tissue comprises an injury, a surgical site, a birth deformity, diseased tissue, or a combination thereof.
Embodiment 124. The method of any of Embodiments 121-123, wherein the soft tissue is of the breast, stomach, abdomen, groin, leg, arm, hand, face, pelvis, uterus, vagina, penis, cervix, brain, nose, ear, eyelid, heart, kidney, liver, bladder, prostate, larynx, trachea, or a combination thereof.
Embodiment 125. The method of any of Embodiments 121-124, wherein the soft tissue comprises breast tissue for breast reconstruction, breast reduction, or breast enlargement.
Embodiment 126. The method of any of Embodiments 121-125 wherein the soft tissue comprises a hernia.
Embodiment 127. The method of any of Embodiments 121-126, wherein the applying comprises affixing the scaffold to the soft tissue of the individual and/or tissue adjacent to the soft tissue of the individual.
Embodiment 128. The method of Embodiment 127, wherein the affixing is further defined as suturing, stapling, or using surgical glue to affix the scaffold to the soft tissue of the individual and/or tissue adjacent to the soft tissue of the individual.
Embodiment 129. The method of Embodiment 128, wherein the suturing is purse-string suturing, continuous suturing, interrupted suturing, buried suturing, deep suturing, or subcutaneous suturing.
Embodiment 130. The method of Embodiment 128 or 129, wherein the suture of the suturing is absorbable.
Embodiment 131. The method of Embodiment 129 or 130, wherein the suture of the suturing is nonabsorbable.
Embodiment 132. A pliable sheet, comprising one or more biodegradable polymers and one or more ECM materials, said sheet comprising a plurality of patterned unit cell structures aligned in adjacent rows of a series of perpendicularly alternating unit cell structures each generally comprising a pore shaped having a central line greater in length than two substantially equal-length lines each perpendicular to opposite ends of the central line, wherein the alternating pattern is configured such that each of the ends of the central line of the pore are generally perpendicular to a central line of a pore of an adjacent unit cell structure.
Embodiment 133. The pliable sheet of Embodiment 132, wherein the unit cell structures are comprised of one or more biodegradable polymers.
Embodiment 134. The pliable sheet of Embodiment 132 or 133, wherein the sheet comprises a coating of one or more ECM materials.
Embodiment 135. The pliable sheet of Embodiment 134, further defined as the unit cell structures comprising a coating of one or more ECM materials.
Embodiment 136. The pliable sheet of any of Embodiments 132-135, wherein the pores are filled with the coating.
Embodiment 137. The pliable sheet of any of Embodiments 132-136, wherein the one or more ECM materials comprises Type I collagen.
Embodiment 138. The pliable sheet of any of Embodiments 132-137, said sheet housed in suitable packaging.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosed subject matter as defined by the appended claims.

1. An artificial support structure comprising one or more biodegradable polymers and one or more extracellular matrix materials, the support structure further comprising:
a plurality of unit patterns, each unit pattern comprising a plurality of filaments, arranged continuously, symmetrically, and regularly thereon, each unit pattern being constituted of edges of a closed shape to thus form a pore at the inside thereof,
wherein the plurality of unit patterns is connected to thus have intersection points with one another and the number of intersection points is the same as the number of edges passing the intersection points.

2. (canceled)

3. (canceled)

4. The artificial support structure of claim 1, wherein the plurality of connected unit patterns form a substantially planar sheet or wherein the plurality of connected unit patterns form a three-dimensional macrostructure.

5. (canceled)

6. The artificial support structure of claim 1, wherein the artificial support structure comprises between 1 and 5 layers.

7. (canceled)

8. (canceled)

9. The artificial support according to claim 1, wherein four-unit patterns are connected to have four intersection points with one another and have four edges passing the four intersection points, and a space surrounded with the four-unit patterns has the same or similar shape as or to each unit pattern.

10. (canceled)

11. The artificial support according to claim 10, wherein short edges of the edges of the closed shape of each unit pattern have the same length as one another, and long edges thereof have the same length as one another, so that the space surrounded with the four-unit patterns has the same shape as each unit pattern.

12. (canceled)

13. (canceled)

14. (canceled)

15. (canceled)

16. (canceled)

17. (canceled)

18. An artificial support structure comprising one or more biodegradable polymers and one or more extracellular matrix materials, the support structure further comprising:
a plurality of unit patterns, each unit pattern comprising a plurality of filaments, arranged repeatedly to constitute columns or rows symmetrical to one another, each unit pattern being constituted of edges of a closed shape to thus form a pore at the inside thereof,
wherein the columns or rows along which the plurality of unit patterns are arranged repeatedly have an Eulerian trail.

19. The artificial support according to claim 18, wherein columns or rows along which the plurality of unit patterns are arranged repeatedly are connected to allow the plurality of unit patterns to have intersection points with the plurality of unit patterns of the neighboring columns or rows.

20. The artificial support according to claim 19, wherein the plurality of unit patterns have the number of intersection points that is the same as the number of edges passing the intersection points.

21. The artificial support according to claim 20, wherein four neighboring unit patterns are connected to have four intersection points with one another and have four edges passing the four intersection points, and a space surrounded with the four-unit patterns has the same or similar shape as or to each unit pattern.

22. The artificial support according to claim 21, wherein short edges of the edges of the closed shape of each unit pattern have the same length as one another, and long edges thereof have the same length as one another, so that the space surrounded with the four-unit patterns has the same shape as each unit pattern.

23. A scaffold, comprising a patterned polymeric substrate having a coating of one or more extracellular matrix (ECM) materials thereon, wherein the pattern of the polymeric substrate comprises adjacent rows of a series of unit cell structures each generally in the shape of the letter “I”, the unit cell structures aligned in the rows of the patterned polymeric substrate in a perpendicularly alternating pattern of the unit cell structures.

24. The scaffold of claim 23 wherein the unit cell structures of the series are further defined as comprising a pore shaped as a central line greater in length than two substantially equal-length lines each perpendicular to opposite ends of the central line, and wherein the alternating pattern is configured such that each of the ends of the central line of the pore are generally perpendicular to a central line of a pore of an adjacent unit cell structure.

25. The scaffold of claim 23, wherein the scaffold is configured as one or more sheets, each comprising a first planar side and a second planar side.

26. The scaffold of claim 25, wherein the scaffold comprises 1, 2, 3, 4, or 5 sheets, or comprises at least or no more than 1, 2, 3, 4, or 5 sheets.

27. The scaffold of claim 25, wherein the multiple sheets are configured such that a planar side of one sheet is adjacent to a planar side of another sheet.

28. The scaffold of claim 23, wherein the scaffold comprises one or more of a defined shape.

29. (canceled)

30. (canceled)

31. The scaffold of claim 23, wherein the scaffold further comprises one or more therapeutic agents.

32. A method of producing the scaffold of claim 23, the method comprising:
(a) three-dimensionally printing the patterned polymeric substrate;
(b) applying the one or more ECM materials to the substrate;
(c) subjecting the substrate to one or more crosslinkers;
(d) optionally washing the substrate; and
(e) subjecting the substrate to conditions less than 15° C. in temperature, optionally wherein the one or more ECM materials and the one or more crosslinkers are mixed together prior to applying to the substrate.

33. A method of reinforcing soft tissue in an individual in need thereof, comprising applying an effective amount of the scaffold of claim 1 to one or more soft tissue sites of the individual.

34. A pliable sheet, comprising one or more biodegradable polymers and one or more ECM materials, said sheet comprising a plurality of patterned unit cell structures aligned in adjacent rows of a series of perpendicularly alternating unit cell structures each generally comprising a pore shaped having a central line greater in length than two substantially equal-length lines each perpendicular to opposite ends of the central line, wherein the alternating pattern is configured such that each of the ends of the central line of the pore are generally perpendicular to a central line of a pore of an adjacent unit cell structure.
